(19) United States
(12) Patent Application Publication
(10) Pub. No.: US 2011/0123452 A1
(43) Pub. Date: May 26, 2011

(54) METAL OLIGOMERS AND POLYMERS AND THEIR USE IN BIOLOGY AND MEDICINE

(75) Inventor: James F. Hainfeld, Shoreham, NY (US)

(73) Assignee: NANOPROBES, INC., Yaphank, NY (US)

(21) Appl. No.: 12/954,167

(22) Filed: Nov. 24, 2010

Related U.S. Application Data
(60) Provisional application No. 61/264,421, filed on Nov. 25, 2009.

(51) Int. Cl. A61K 38/06; A61K 31/715; A61K 49/00; C07H 23/00; A61P 43/00; A61P 35/00; A61P 35/02; A61P 37/00; C07K 5/093; A61K 31/28; C01G 9/00

(52) U.S. Cl. 424/9.1; 514/21.9; 514/54; 536/121; 530/331; 514/495; 556/110

(57) ABSTRACT

Described herein are a class of metal oligomers and polymers that contain both metals and organic groups. Said oligomers and polymers have utility in many applications including biomedical imaging, radiation therapy, drug delivery, and in vitro analytical techniques, such as fluorescence and phosphorescence.

### CROSS REFERENCE

This application claims priority to U.S. Provisional Application No. 61/264,421, entitled, “Metal Oligomers and Polymers and Their Use in Biology and Medicine,” filed on Nov. 25, 2009, the contents of which are incorporated by reference in its entirety.

### BACKGROUND OF THE INVENTION

Nanoparticles containing metals have become increasingly popular and are finding new applications in many areas and disciplines. For example, they have been used as reporter materials in diagnostics using light absorption and scattering, as dense markers for light and electron microscopy, as heat absorbers for detection and hyperthermia therapy, as materials to enhance radiation therapy, as medical imaging contrast agents, as platforms for Surface Enhanced Raman Spectroscopy (SERS) sensitive detectors, as x-ray absorbers to enhance radiotherapy, as drug delivery vehicles, as components in nanowires and nanodevices, as food additives, magnetic nanoparticles for separations and hyperthermia, as highly fluorescent quantum dots, as DNA carriers for transfection, and many other uses.

### SUMMARY OF THE INVENTION

Described herein are metal oligomers and polymers, that contain both metal atoms and organic groups, which have desirable properties for such applications including medical imaging, radiation enhancement, and use as drugs or drug carriers, as well as the synthesis of such metal oligomers and polymers.

Presented herein is a composition having the structure of Formula (II):

X-M₁-Y_M₂ₙ; Formula (II)

wherein:

M₁ and M₂ are each independently a metal atom selected from scandium, titanium, vanadium, chromium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum, gold, gadolinium, aluminum, gallium, indium, tin, thallium, lead, bismuth, magnesium, calcium, strontium, barium, lithium, sodium, potassium, boron, silicon, phosphorus, germanium, arsenic, antimony, and combinations thereof;

X and Y are each independently selected from S(R₁) or S(R₂)—S, S—S, or P(R₃)₃;

R₁, R₂, and R₃ are each independently an organic group;

n is an integer from 2 to about 2000; and a pharmaceutically acceptable buffer.

In one embodiment is the composition having the structure of Formula (II) wherein M₁ and M₂ are both the same. In one embodiment, M₁ and M₂ are selected from gold, platinum, osmium, iridium, thallium, lead, bismuth, tungsten, silver, palladium, and molybdenum. In another embodiment, M₁ and M₂ are both gold. In a further embodiment, M₁ and M₂ are both different.

In one aspect is a composition having the structure of Formula (I):

X—Au—Y—Auₙ; Formula (I)

wherein:

X and Y are each independently selected from S(R₁) or S(R₂)—S, S—S, or P(R₃)₃;

R₁ and R₂ are each independently an organic group;

n is an integer from 2 to about 2000; and pharmaceutically acceptable buffer.

In one embodiment is a composition of Formula (I) having the structure of Formula (IA):

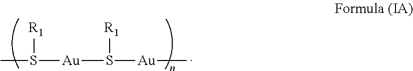

In another embodiment is a composition having the structure of Formula (I) wherein Y is S(R₂)—S. In yet another embodiment, the composition further comprises a pharmaceutically acceptable buffer.

In a further embodiment is a composition of Formula (I) having the structure of Formula (IB):

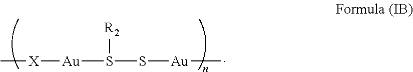

In yet another embodiment is a composition having the structure of Formula (IA) or (IB) wherein X is S(R₁). In another embodiment, is the composition having the structure of Formula (IB) wherein X is S(R₂)—S. In yet another embodiment, the composition having the structure of Formula (IB) further comprises a pharmaceutically acceptable buffer.

In another embodiment is the composition having the structure of Formulas (IC) or (ID):

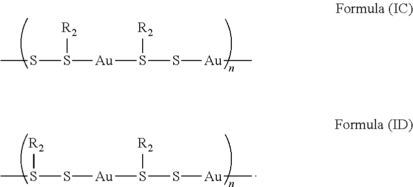

In yet another embodiment, is a composition having the structure of Formulas (IA), (IB), (IC), or (ID) wherein the organic group comprises a peptide fragment, a peptide, an antibody fragment, an antibody, a single chain antibody fragment, a single chain antibody, a protein fragment, a protein, a lipid fragment, a lipid, a carbohydrate fragment, a carbohydrate, an aptamer fragment, an aptamer, a nucleic acid fragment, a nucleic acid, a thiol-containing moiety, a porphyrin fragment or a porphyrin. In one embodiment, R₁ and/or R₂ is a peptide fragment. In another embodiment, the peptide fragment is a glutathione fragment. In a further embodiment, R₁ and/or R₂ is a carbohydrate fragment. In yet a further embodiment, R₁ and/or R₂ is a thiosugar, such as thioglucose, thiogalactose, or thiosucrose. In yet a further embodiment, R₁ and/or R₂ is a thioglucose fragment. In yet another embodiment, the organic group comprises glutathione, thioglucose, dithiothreitol, lipoic acid, dihydrolipoic acid, lipoamide, dihydrolipoamide, thiocholesterol, thiopropionic acid, cysteine, thiophenol, mercaptoethylamine, mercaptoethanol, thiol-containing polyalkylene glycol, dodecanethiol in combination with tween 20, and dithiobis[succinimidyl propionate] or fragments thereof. In one embodiment, n is an integer from 2 to about 20. In one embodiment, n is an integer from about 20 to about 100. In one embodiment, n is an integer from about 100 to about 1000. In another embodiment, n is an integer from about 1000 to about 2000. In one embodiment, each R₁ is the same. In a further embodiment, each R₁ is different. In one embodiment, each R₂ is the same. In another embodiment, each R₂ is different.

Described herein is a method for biological imaging of a biological system comprising, administering to the biological system a dose of a composition having the structure of Formula (I), (IA), (IB), (IC), (ID), or (II) and subjecting the biological system to an imaging technique. In another embodiment, is a method for biological imaging of a biological system wherein the composition having the structure of Formula (I), (IA), (IB), (IC), (ID), or (II) further comprises a pharmaceutically acceptable buffer.

Also described herein is a method of sensitizing a biological system to the effects of radiation, comprising administering to the biological system an effective amount of the composition(s) described herein, and exposing the biological system to a source of radiation. In one embodiment, the composition described herein comprises a tumor-targeting moiety. In another embodiment, the biological system is a patient in need of radiotherapy for the treatment of cancer or other neoplastic disease. One embodiment provides the composition of Formula (I), (IA), (IB), (IC), (ID), or (II), wherein the composition has a whole body clearance of greater than about 90% after one week. In another embodiment provides the composition of Formula (I), (IA), (IB), (IC), (ID), or (II), wherein the composition has a whole body clearance of greater than about 95% after one week.

In another embodiment, the composition having the structure of Formula (I), (IA), (IB), (IC), (ID), and (II) comprises a plaque-targeting moiety. In one embodiment, the compositions described herein comprises a DNA-targeting moiety. In yet another embodiment, the tumor-targeting moiety comprises a tumor-specific antibody. In yet another embodiment, the R₁ or R₂ groups described herein comprises

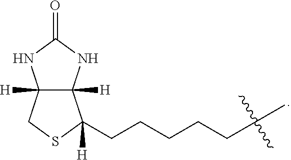

In another embodiment, the tumor-specific antibody is non-covalently attached through a biotin-avidin complex. In a further embodiment, the composition described herein comprises at least one R₁ and/or R₂ group comprising a DNA-binding moiety selected from ethidium bromide, Hoeschst dyes or acridines. In another embodiment, is a composition comprising a compound of Formula (I) comprising a trifluoroaziridine group. In another embodiment, the composition comprises a sensitizing moiety selected from porphyrin, photophrin, texaphyrin, phthalocyanine, or benzophenone.

In one embodiment, is a method of brachytherapy, comprising implanting a dose of the composition of Formula (II), wherein the composition comprises a ¹²⁵I isotope, and ¹⁶⁹Yb isotope or ¹⁰³Pd isotope.

Also described herein is a composition having the structure shown in Formula (IE):

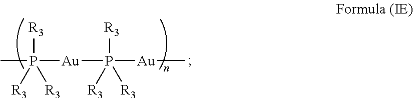

wherein each R₄ is independently an organic group.

In one embodiment is a composition having the structure of Formula (I), (IA), (IB), (IC), (ID), (IE), or (II) wherein each R₁, R₂, or R₃ group is independently selected from an alkyl group, an aryl group, a heteroaryl group, a heterocyclo group, a sugar, a peptide, or a poly(alkyleneglycol) group.

One embodiment provides the compositions described herein, wherein the composition exhibits fluorescence. Another embodiment provides the composition, wherein the composition exhibits low loss of fluorescence upon illumination.

### DETAILED DESCRIPTION

### Poor Clearance

As useful as nanoparticles may be, they have limitations for some applications. For example, although intravenously administered 15 nm gold nanoparticles coated with polyethylene glycol have a blood half-life of hours and are considered “stealth nanoparticles” which avoid rapid liver clearance, they do not readily clear the animal well, and measurements show that after one week and even one month, animals still retain about 48% of the injected gold. Therefore, such an agent may be good for x-ray imaging animals, but is unfavorable for general human use, especially in screening of asymptomatic patients, due to this poor clearance. Smaller gold nanoparticles, e.g., approximately 2-5 nm, filter through the kidneys and have better clearance profiles, but even these typically exhibit retention of about 20% of the injected gold after one week. Further, these nanoparticles are colored and at higher doses significantly color the skin, e.g., brown-black, purple, or red, immediately after injection; due to the extended retention, some of this color can remain even weeks later. While this may not be harmful, it can be cosmetically objectionable.

Poor Diffusion

Nanoparticles also poorly diffuse into tissue. Large particles, approximately 500-1000 nm have very poor tissue penetration, and similarly, particles approximately 50-500 nm also show, for example, limited penetration into many tumors, even though the angiogenic vasculature is leaky. Entry into cells can also have a negative dependence on size, among other factors. Large materials, like nanoparticles, cannot directly cross the cell membrane as can some small molecules, but may enter the cell via endocytosis, thus terminating in the endosome or lysosome. For drug delivery, where many targets are nuclear or cytoplasmic, the nanoparticle or its cargo must escape the endosome, thus posing an additional barrier. Nuclear pores exclude many nanoparticles from access to the nucleus.

Nanoparticle Toxicity

In some embodiments, nanoparticle toxicity may also be problematic. The highly useful fluorescent quantum dots are generally made out of cadmium or lead, thus prohibiting their use in humans. Since all materials are toxic at some level, even other more benign particles have toxicity limits. The target organ of toxicity may also vary depending on the nanoparticle's size and coating. Carbon nanotubes have now been shown to be quite toxic, and even some gold nanoparticles have unacceptable toxicities for human use. There can be additional problems, such as Argyria, where silver containing nanoparticles permanently color the skin blue.

With respect to fluorescence, various organic ring containing compounds have served well, but exhibit bleaching, or loss of fluorescence, upon illumination. Brightness or quantum yield is also limited. Quantum dots improve on these properties, having less bleaching and being brighter, but also have a number of disadvantages for many applications, including large size (5-20 nm), blinking and toxicity. Large polymers such as polyethylene glycol (PEG) are commonly attached to their surface to obtain water solubility and biocompatibility, but these can considerably increase the overall size.

Nanoparticles, e.g., gold nanoparticles, have been used to enhance the effects of radiation, due to absorption of x-rays or other radiations, and subsequent local deposition of this energy or reaction products in the local region. These have experimentally been shown to improve radiotherapy of tumors in animals. However, nanoparticles have various restrictions, such as limitation of tumor penetration, diffusion, cell entry, cytoplasmic and nuclear delivery.

### Metal Oligomers and Polymers

Disclosed herein are metal oligomers and polymers that have properties favorable for use in biology and medicine. Metals are oligomerized or polymerized by organic ligands that bond to metal atoms, as well as by smaller metal oligomers and polymers that associate further through metal-metal and/or ligand-ligand interactions. Oligomer or polymer properties such as size, structure, solubility, biocompatibility, pharmacokinetics, toxicity, and stability are designed and controlled through the selection of appropriate metal centers or ligands. These oligomers and polymers demonstrate properties that are useful in applications such as medical imaging, where properties are chosen to optimize such characteristics as blood half-life, tumor, organ, or tissue targeting, and clearance. The oligomers and polymers disclosed herein also have utility as detection reporters, using for example, visible light, infrared, ultraviolet (UV), or x-rays. Another embodiment provides metal oligomers and polymers with fluorescent and phosphorescent properties, and having utility as sensitive detectors.

One embodiment provides a composition having the structure shown in Formula (I):

X—Au—Y—Auₙ Formula (I)

wherein X and Y are each independently selected from —S(R₁)—, —S(R₂)—S—, —S—S—, P(R₃)₃, or N(R₄)₃ and wherein each R₁, R₂, R₃ or R₄ group is an organic group, n is an integer from 4 to about 2000; and a pharmaceutically acceptable buffer.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the present disclosure will become apparent from this detailed description. All references cited herein, including patents, patent applications, and publications, are hereby incorporated by reference to the extent they are relevant for the purposes described herein.

### Certain Terminology

It is to be understood that the description presented herein is exemplary and explanatory only and are not restrictive of any subject matter claimed. In this document, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Definition of standard chemistry terms may be found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein.

An “alkoxy” group refers to a (alkyl)O— group, where alkyl is as defined herein.

An “alkyl” group refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group that has at least one carbon-carbon double bond, and an “alkyne” moiety refers to a group that has at least one carbon-carbon triple bond. The alkyl moiety, whether saturated or unsaturated, may be branched, straight chain, or cyclic. Depending on the structure, an alkyl group can be a monoradical or a diradical (i.e., an alkylene group).

As used herein, C₁-Cₓ includes C₁-C₂, C₁-C₃ . . . C₁-Cₓ.

In some embodiments, the “alkyl” moiety has 1 to 30 carbon atoms (whenever it appears herein, a numerical range such as “1 to 30” refers to each integer in the given range; e.g., “1 to 30 carbon atoms” means that, in other embodiments, the alkyl group has 1 carbon atom, 2 carbon atoms, 3 carbon atoms, etc., up to and including 30 carbon atoms, although the present definition also covers the occurrence of the term “alkyl” where no numerical range is designated). In other embodiments, the alkyl group of the compounds described herein is designated as “C₁-C₄ alkyl” or similar designations. By way of example only, “C₁-C₄ alkyl” indicates that there are one to four carbon atoms in the alkyl chain, i.e., the alkyl chain is selected from among methyl, ethyl, propyl, iso-propyl, n-butyl, iso-butyl, sec-butyl, and t-butyl. Thus C₁-C₄ alkyl includes C₁-C₂ alkyl and C₁-C₃ alkyl. Alkyl groups can be substituted or unsubstituted. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, heptyl, octyl, nonyl, decyl, undecyl, dodecyl, tridecyl, tetradecyl, pentadecyl, hexadecyl, heptadecyl, octadecyl, and nonadecyl.

As used herein, the term “non-cyclic alkyl” refers to an alkyl that is not cyclic (i.e., a straight or branched chain containing at least one carbon atom). Non-cyclic alkyls can be fully saturated or can contain non-cyclic alkenes and/or alkynes. Non-cyclic alkyls can be optionally substituted.

The term “alkylamine” refers to the —N(alkyl)ₓH_{y} group, where x and y are selected from among x=1, y=1 and x=2, y=0. When x=2, the alkyl groups, taken together with the N atom to which they are attached, can optionally form a cyclic ring system.

An “amide” is a chemical moiety with the formula —C(O)NHR or —NHC(O)R, where R is selected from among alkyl, cycloalkyl, aryl, heteroaryl (bonded through a ring carbon) and heteroalicyclic (bonded through a ring carbon). In some embodiments, an amide moiety forms a linkage between an amino acid or a peptide molecule and a compound described herein, thereby forming a prodrug. Any amine, or carboxyl side chain on the compounds described herein can be amidified. The procedures and specific groups to make such amides are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference to the extent it is relevant for the purposes described herein.

The term “aromatic” refers to a planar ring having a delocalized π-electron system containing 4n+2π electrons, where n is an integer. In other embodiments, aromatic rings are formed by five, six, seven, eight, nine, or more than nine atoms. In further embodiments, aromatics are optionally substituted. The term “aromatic” includes both carbocyclic aryl (e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups. As used herein, “Π-Π interactions” are caused by intermolecular overlapping of p-orbitals in Π-conjugated systems such that they become stronger as the number of Π-electrons increases.

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

The term “carbocyclic” refers to a compound which contains one or more covalently closed ring structures, and that the atoms forming the backbone of the ring are all carbon atoms. The term thus distinguishes carbocyclic from heterocyclic rings in which the ring backbone contains at least one atom which is different from carbon.

The term “bond” or “single bond” refers to a chemical bond between two atoms, or two moieties when the atoms joined by the bond are considered to be part of larger substructure.

As used herein, “non-covalent” interactions refers to interactions that are generally weaker than covalent bonds and include Coulomb interactions, hydrogen bonds, ion-ion interactions, ion-dipole interactions, dipole-dipole interactions, cation-π interactions, π-π interactions, van der Waals forces, London Dispersion Forces, hydrophobic effects and metal ligand coordination (Steed, J. W. Atwood, J. L. Supramolecular Chemistry; Wiley & Sons: Chichester, 2000; Hoeben F. J. M., Jonkhejim, P.; Meijer, E. W., Schenning, A. P. H. J, Chem. Rev. 2005, 105, 1491-1546). Covalent bonds normally have a homolytic bond dissociation energy that ranges between about 100 kJmol⁻¹ to about 420 kJmol⁻¹.

As used herein, “amphiphatic molecules” refers to molecules that contain both a hydrophilic moiety and a hydrophobic moiety. In reference to amphiphatic molecules, a hydrophilic group is also referred herein to an environmental group.

The term “cycloalkyl” refers to a monocyclic or polycyclic radical that contains only carbon and hydrogen, and may be saturated, partially unsaturated, or fully unsaturated. Cycloalkyl groups include groups having from 3 to 10 ring atoms. Illustrative examples of cycloalkyl groups include the following moieties:

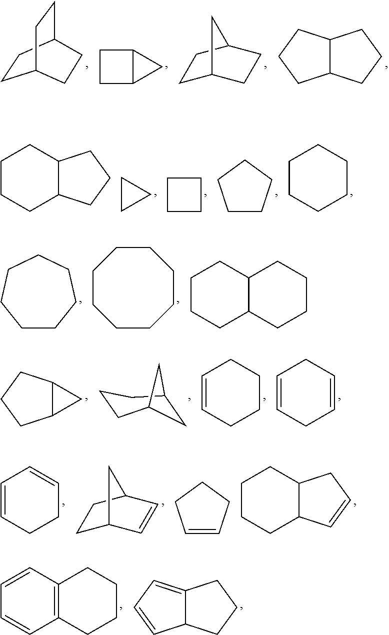

and the like. Depending on the structure, an cycloalkyl group can be a monoradical or a diradical (e.g., an cycloalkylene group).

As used herein, the term “carbocycle” refers to a ring, wherein each of the atoms forming the ring is a carbon atom. Carbocylic rings can be formed by three, four, five, six, seven, eight, nine, or more than nine carbon atoms. Carbocycles can be optionally substituted.

The term “ester” refers to a chemical moiety with formula —COOR, where R is selected from among alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference to the extent it is relevant for the purposes described herein.

The term “halo” or, alternatively, “halogen” or “halide” means fluoro, chloro, bromo or iodo.

The terms “haloalkyl,” “haloalkenyl,” “haloalkynyl” and “haloalkoxy” include alkyl, alkenyl, alkynyl and alkoxy structures in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another. The terms “fluoroalkyl” and “fluoroalkoxy” include haloalkyl and haloalkoxy groups, respectively, in which the halo is fluorine. In certain embodiments, haloalkyls are optionally substituted.

As used herein, the terms “heteroalkyl” “heteroalkenyl” and “heteroalkynyl” include optionally substituted alkyl, alkenyl and alkynyl radicals in which one or more skeletal chain atoms are selected from an atom other than carbon, e.g., oxygen, nitrogen, sulfur, silicon, phosphorus or combinations thereof.

The term “heteroatom” refers to an atom other than carbon or hydrogen. Heteroatoms are typically independently selected from among oxygen, sulfur, nitrogen, silicon and phosphorus, but are not limited to these atoms. In embodiments in which two or more heteroatoms are present, the two or more heteroatoms can all be the same as one another, or some or all of the two or more heteroatoms can each be different from the others.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can form part of a ring system.

As used herein, the term “ring system” refers to two or more rings, wherein two or more of the rings are fused. The term “fused” refers to structures in which two or more rings share one or more bonds.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. The polycyclic heteroaryl group may be fused or non-fused. Illustrative examples of heteroaryl groups include the following moieties:

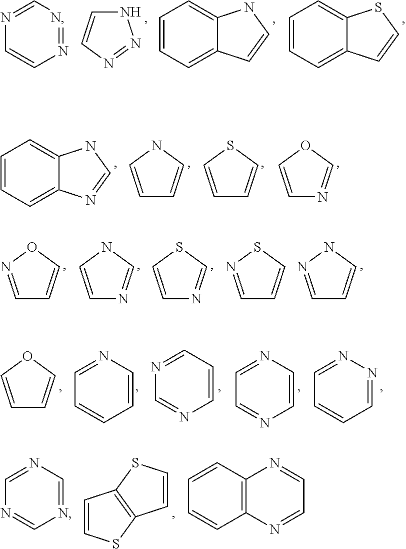

and the like. Depending on the structure, a heteroaryl group can be a monoradical or a diradical (i.e., a heteroarylene group).

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

An “isocyanato” group refers to a —NCO group.

The term “moiety” refers to a specific segment or functional group of a molecule. Chemical moieties are often recognized chemical entities embedded in or appended to a molecule.

As used herein, the term “O-carboxy” refers to a group of formula RC(═O)O—.

As used herein, the term “C-carboxy” refers to a group of formula —C(═O)OR.

As used herein, the term “acetyl” refers to a group of formula —C(═O)CH₃.

As used herein, a “xanthate” refers to RO—C(═S)—SR.

As used herein, a “thiocarbamate” refers to RO—C(═S)—NR₂.

As used herein, a “urea” refers to R₂N—C(═O)—NR₂.

As used herein, a “thiourea” refers to R₂N—C(═S)—NR₂.

As used herein, the term “trihalomethanesulfonyl” refers to a group of formula X₃CS(═O)₂— where X is a halogen.

As used herein, the term “cyano” refers to a group of formula —CN.

A “selenol” refers to R—SeH.

A “selenolate” refers to R—Se⁻, which is the deprotonated form of a selenol.

A “diselane” refers to R—Se—Se—R.

A “thiol” refers to R—SH.

A “thiolate” refers to R—S⁻, which is the deprotonated form of a thiol.

A “sulfate” refers to a —OS(═O)₂—OR.

A “sulfinyl” group refers to a —S(═O)—R.

A “sulfonyl” group refers to a —S(═O)₂—R.

A “thioalkoxy” group refers to a —S-alkyl group.

As used herein, the term “S-sulfonamido” refers to a group of formula —S(═O)₂NR₂.

As used herein, the term “N-sulfonamido” refers to a group of formula RS(═O)₂NH—.

As used herein, the term “sulfate” refers to a group of the formula —OS(═O)₂OR.

As used herein, the term “phosphate” refers to a groups of the formula —OP(═O)₂OR.

As used herein, the term “phosphonate” refers to a groups of the formula —OP(═O)OR₂.

As used herein, the term “phosphinate” refers to a groups of the formula —OP(═O)R₂.

As used herein, the term “O-carbamyl” refers to a group of formula —OC(═O)NR₂.

As used herein, the term “N-carbamyl” refers to a group of formula ROC(═O)NH—.

As used herein, the term “C-amido” refers to a group of formula —C(═O)NR₂.

As used herein, the term “N-amido” refers to a group of formula RC(═O)NH—.

As used herein, the term “absorptivity” refers to the ability of a substance to impede transmittance of light of a given wavelength. This property can be described in terms of an extinction coefficient, a reduction in the transmission of light through a sample (regardless of mechanism of action), or by the ability of a substance to absorb light (again, regardless of mechanism).

“Antioxidants” include, for example, butylated hydroxytoluene (BHT), sodium ascorbate, ascorbic acid, sodium metabisulfite and tocopherol. In certain embodiments, antioxidants enhance chemical stability where required.

The term “acceptable” or “pharmaceutically acceptable” with respect to a formulation, composition or ingredient, as used herein, means having no persistent detrimental effect on the general health of the subject being treated.

As used herein, “amelioration” of the symptoms of a particular disease, disorder or condition by administration of a particular pharmaceutical composition refers to any lessening of severity, delay in onset, slowing of progression, or shortening of duration, whether permanent or temporary, lasting or transient that can be attributed to or associated with administration of the composition.

As used herein, the term “antibody” refers to any polypeptide that contains an immunoglobulin hypervariable (CDR) region antigen binding domain. For example, the antibody can be a monovalent antibody, a divalent antibody, a Fab fragment, a single-chain Fᵥ, a monoclonal, or polyclonal antibody.

The term “bound,” as used herein refers to one or more associations, interactions, or bonds that are covalent or non-covalent (including ionic bonds, hydrogen bonds, and van der Waals interactions).

The term “buffer” as used herein refers to an agent that adjusts the pH of a solution. The function of a buffer or buffering agent is to drive an acidic or basic solution to a certain pH state and prevent a change in this pH.

The term “carrier,” as used herein, refers to relatively nontoxic chemical compounds or agents that facilitate the transport of metal oligomers and/or polymers into vasculature, tissues, or cells.

The terms “co-administration” or the like, as used herein, are meant to encompass administration of the selected therapeutic agents to a single patient, and are intended to include treatment regimens in which the agents are administered by the same or different route of administration or at the same or different time.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution.

As used herein, “EC₅₀” refers to a dosage, concentration or amount of metal oligomers and/or polymers that elicits 50% of a maximal effect that is induced, provoked, or potentiated by the metal oligomers and/or polymers.

The term “effective amount,” refers to the amount of metal oligomers and polymers that is required to obtain a therapeutic or diagnostic effect. In other embodiments, it is also the amount of metal oligomers and polymers required to obtain a therapeutic or diagnostic effect in combination with a therapeutically effective dose of radiation. A “therapeutically effective amount,” as used herein, refers to an amount of metal oligomers and polymers sufficient to allow detection of a target when the metal oligomers and polymers are provided to the therapeutic target and the therapeutic target is exposed to a therapeutically effective dose of radiation or sufficient to relieve to some extent one or more of the pathological indicia associated with the therapeutic target when exposed to a therapeutically effective dose of radiation. The result in some embodiments is a reduction and/or alleviation of the signs, symptoms, or causes of a disease, or any other desired alteration of a biological system. For example, an “effective amount” for therapeutic uses is the amount of metal oligomers and polymers as disclosed herein required to provide a clinically significant decrease in disease symptoms or other pathological indicia without undue adverse side effects. It is understood that “an effective amount” or “a therapeutically effective amount” can vary from subject to subject, due to variation in therapeutic target size, shape, depth, composition, as well as systemic factors such as circulation, metabolism, age, weight, general condition of the subject, the severity of the therapeutic target-associated condition being treated, and the judgment of the prescribing physician.

As used herein, the term “infrared” refers to any wavelength between about 700 to about 1100 nm.

The term “metal oligomers or polymer,” as used herein refers, in some embodiments, to an oligomer or polymer that has a core mass which is at least about 20% metallic by weight. In other embodiments, the core mass of the metal oligomer or polymer is at least about 30% metallic by weight. In some embodiments, the oligomer or polymer has a core mass which is at least about 40% metallic by weight.

The term “non-target,” as used herein, refers to a biological substrate outside of a volume or surface occupied by a therapeutic target. Such therapeutic targets include, but are not limited to, a tumor, a volume of infected tissue, a volume of degenerated tissue, a volume of inflamed tissue, a blood clot, or a region of plaque.

The term “pharmaceutical combination” as used herein, means a product that results from the mixing or combining of more than one active ingredient and includes both fixed and non-fixed combinations of the active ingredients. The term “fixed combination” means that the active ingredients, e.g. metal oligomers and/or polymers described herein and a co-agent, are both administered to a patient simultaneously in the form of a single entity or dosage. The term “non-fixed combination” means that the active ingredients, e.g. metal oligomers and polymers described herein and a co-agent, are administered to a patient as separate entities either simultaneously, concurrently or sequentially with no specific intervening time limits, wherein such administration provides effective levels of the two agents in the body of the patient. The latter also applies to cocktail therapy, e.g. the administration of three or more active ingredients.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 100,000, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

A “subject,” as referred to herein, can be any verterbrate, though preferably a mammal (e.g., a mouse, rat, cat, guinea pig, hamster, rabbit, zebrafish, dog, non-human primate, or human) unless specified otherwise.

The term “therapeutic target” refers to a biological substrate (e.g., a tumor, a region of infected tissue, or a region of atheromatous plaque) that is to be acted upon by metal oligomers and/or polymers as described herein.

The terms “treat,” “treating” or “treatment,” as used herein, include alleviating, abating or ameliorating symptoms or pathological indicia of a therapeutic target-associated disease or condition, (e.g., breast tumor-breast cancer) preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

### Metal Oligomer and Polymer Compositions

Presented herein are metal oligomers and polymers that have properties favorable for use in biology and medicine. In some embodiments, metals are oligomerized or polymerized by organic ligands that bond to metal atoms, as well as by smaller metal oligomers and polymers that associate further through metal-metal and/or ligand-ligand interactions. Oligomer or polymer properties such as size, structure, solubility, biocompatibility, pharmacokinetics, toxicity, and stability are designed and controlled through the selection of appropriate metal centers or ligands.

In one aspect are metal atoms which coordinate with various other atoms and compounds to form metal-non-metal bonds. Under the proper conditions, it has been found that this basic property can be controlled to produce materials with multiple metal atoms and multiple organic groups. The metal atom is capable of forming at least two bonds with another type of atom. In other embodiments, if that other atom also forms two or more bonds with a metal atom, then the process can be repeated, and oligomers or polymers will result. The polymerization process may also progress by non-covalent interaction between the components. The bonding patterns of particular atoms may depend on their oxidation state. One embodiment provides a gold atom in the +1 oxidation state that has two bonds it can form with other atoms. Sulfur forms three bonds, one bond to a carbon atom and two bonds to two gold atoms.

In another aspect is a composition having the structure of Formula (II):

X-M₁-Y-M₂ₙ; Formula (II)

wherein:

M₁ and M₂ are each independently a metal atom;

X and Y are each independently selected from S(R₁), S(R₂)—S, S—S, or P(R₃)₃;

R₁, R₂, and R₃ are each independently an organic group;

n is an integer from 2 to about 2000; and a pharmaceutically acceptable buffer.

In one embodiment, is the composition of Formula (II) wherein M₁ and M₂ are each independently selected from scandium, titanium, vanadium, chromium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum, gold, gadolinium, aluminum, gallium, indium, tin, thallium, lead, bismuth, magnesium, calcium, strontium, barium, lithium, sodium, potassium, boron, silicon, phosphorus, germanium, arsenic, antimony, and combinations thereof. In another embodiment, M₁ and M₂ are each gold. In a further embodiment, M₁ and M₂ are both the same. In yet a further embodiment, M₁ and M₂ are different. In some embodiments, the metal atom (M₁ and/or M₂) of the metal oligomers and polymers is selected from Fe, Zn, Mn, Cr, Cu, Ca, and Ni. In other embodiments, the metal atom is Zn. In a further embodiment is the composition of Formula (II) wherein the organic group of R₁, R₂, and R₃ comprises antibodies, drugs, prodrugs, peptides, amino acids, ethylene glycol units, other polymers, proteins, carbohydrates, lipids, nucleic acids, other biomolecules, synthetic molecules, or organic group fragments. In another embodiment, groups such as antibodies, drugs, prodrugs, peptides, ethylene glycol units, other polymers, proteins, carbohydrates, lipids, nucleic acids, other biomolecules, synthetic molecules, are attached to R₁, R₂, and/or R₃ of the metal oligomer and/or polymer. In some embodiments, the organic groups of R₁, R₂, and R₃ include for example, anti-bacterial compounds, anti-viral compounds, anti-fungal compounds, anti-protozoan compounds, anti-histamines, immunomodulatory compounds, anesthetic compounds, steroidal antiinflammatory agents, antiinflammatory analgesics, chemotherapeutic agents, hormones, immunosuppressants, protease inhibitors, and aldose reductase inhibitors, corticoid steroids, immunosuppressives, cholinergic agents, anticholinesterase agents, a peptide fragment, an antibody fragment, a single chain antibody fragment, a protein fragment, a lipid fragment, a carbohydrate fragment, an aptamer fragment, an aptamer, a nucleic acid fragment, a thiol-containing moiety, a porphyrin fragment or a porphyrin.

Nucleic acids suitable for use as organic groups or fragments thereof include oligonucleotides and polynucleotides formed of DNA and RNA, and analogs thereof, which have selected sequences designed for hybridization to complementary targets (e.g., antisense sequences for single- or double-stranded targets), or for expressing nucleic acid transcripts or proteins encoded by the sequences. Analogs include charged and preferably uncharged backbone analogs, such as phosphonates (preferably methyl phosphonates), phosphoramidates (N3′ or N5′), thiophosphates, uncharged morpholino-based polymers, and protein nucleic acids (PNAs). In some embodiments, such molecules are used in a variety of therapeutic regimens, including enzyme replacement therapy, gene therapy, and anti-sense therapy, for example.

Peptides herein include, but should not be limited to, effector polypeptides, receptor fragments, and the like. Examples include peptides having phosphorylation sites used by proteins mediating intra-cellular signals. Examples of such proteins include, but are not limited to, protein kinase C, RAF-1, p21Ras, NF-κB, C-JUN, and cytoplasmic tails of membrane receptors such as IL-4 receptor, CD28, CTLA-4, V7, and MHC Class I and Class II antigens.

In some embodiments, when a peptide or peptide fragment is used herein, the synthesis is achieved either using an automated peptide synthesizer or by recombinant methods in which a polynucleotide encoding a fusion peptide is produced.

In a further embodiment, the organic group is attached to the metal atom via a linker. In further embodiments, the linker contains a sulfur atom. In further embodiments, the linker comprises a polyalkylene group such as, for example only, a PEG group. In some embodiments, is the composition having the structure of Formula (II) wherein M₁ and M₂ are linked together. In further embodiments, the metal oligomer and polymer having the structure of Formula (II) is preformed. In a further embodiment, the metal oligomer and polymer having the structure of Formula (II) is formed in situ.

In one embodiment, is a composition having the structure of Formula (II) wherein n is an integer from 2 to about 2000. In another embodiment, n is an integer from about 5 to about 1500. In another embodiment, n is an integer from about 10 to about 1200. In another embodiment, from about 50 to about 1000. In a further embodiment, from about 100 to about 500. In yet another embodiment, n is about 5, about 10, about 20, about 25, about 50, about 75, about 100, about 150, about 200, about 300, about 400, about 500, about 600, about 700, about 800, about 900, about 1000, about 1100, about 1200, about 1300, about 1400, about 1500, about 1600, about 1700, about 1800, about 1900, or about 2000.

In a further embodiment, is the composition having the structure of Formula (II) wherein X and Y are each independently selected from S(R₁), S(R₂)—S, and S—S. In other embodiments, are compositions having the structure of Formula (II) wherein X and Y are both P(R₃)₃. Similarly, other atoms that bond with gold atoms can be used, e.g., phosphorus and nitrogen. One embodiment provides phosphine complexes with one gold atom associated with the phosphorous. Another embodiment provides a metal oligomer or polymer with the structure (R₃P—Au—PR₃). Another embodiment provides larger oligomers and polymers having phosphine complexes with more than one gold atom associated with the phosphorous.

In one embodiment, are compositions comprising a compound having a structure of Formula (II) and a pharmaceutically acceptable buffer. In other embodiments, the compositions comprise a physiologically compatible buffer, such as Hank's solution, Ringer's solution, or physiological saline buffer. In other embodiments, the compositions comprise a pharmaceutically acceptable buffer at a concentration which results in an increase in stability of the metal oligomer/polymer compound. To this end, in some embodiments, variations in formulation composition include, but are not limited to, variations in pH within an acceptable range for storage of a biologically active metal oligomer/polymer. In some embodiments, the pharmaceutically acceptable buffer is at a concentration effective to maintain the pH of the composition within a range of about 6 to about 9. In other embodiments, the pharmaceutically acceptable buffer is at a concentration effective to maintain the pH of the composition within a range of about 6.5 to about 8.5, about 7 to about 8. In further embodiments, the pharmaceutically acceptable buffer is at a concentration effective to maintain the pH of the composition at a pH of about 7.7. In other embodiments, the buffer provides a pH of the concentration of about 7.0, about 7.1, about 7.2, about 7.3, about 7.4, about 7.5, about 7.6, about 7.7, about 7.8, about 7.9, and about 8.0.

Buffers described herein include, but are not limited to Tris, sodium phosphate, potassium phosphate, HEPES, ACES, TRIS, TES, MOPS, Tricine, Bicine, TAPS, PBS, and saline sodium citrate. In other embodiments, the compositions described herein comprise a compound described herein and a phosphate buffered saline (PBS), wherein the pH is adjusted from about pH 7 to about 8. The buffers described herein are included in an amount required to maintain pH of the composition in an acceptable range, such as is suitable for parenteral administration.

In other embodiments, the compositions described herein are formulated for parenteral administration. In yet other embodiments, the parenteral injections comprise appropriate formulations which include but are not limited to aqueous or nonaqueous solutions; such solutions include physiologically compatible buffers and/or excipients.

In other embodiments, the buffering agent incorporated in the formulation of the compositions is selected from those capable of buffering the preparation to a pH within a physiologically tolerable range for administration to a patient.

Also described herein are compositions having the structure of Formula (I):

X—Au—Y—Auₙ; Formula (I)

wherein:

X and Y are each independently selected from S(R₁) or S(R₂)—S, S—S, or P(R₃)₃;

R₁ and R₂ are each independently an organic group; n is an integer from 2 to about 2000; and

a pharmaceutically acceptable buffer.

In one embodiment, is the composition comprising the compound having the structure of Formula (I) wherein the pharmaceutically acceptable buffer is at a concentration effective to maintain the pH of the composition within the range of about 6.5 to about 8.5. In another embodiment, the pharmaceutically acceptable buffer is at a concentration effective to maintain the pH of the composition within the range of about 7 to about 8.

The composition of any of claims 1-30 wherein the pharmaceutically acceptable buffer is a phosphate buffer. In one embodiment, X and Y are both the same. In another embodiment, X and Y are S(R₁). In one embodiment, is a composition having the structure of Formula (I) wherein X is S(R₁) and Y is S(R₂)—S. In yet another embodiment, X and Y are both S(R₂)—S. In one embodiment, X is S—S and Y is S(R₂)—S. In another embodiment, X is S—S and Y is S(R₁). In yet another embodiment, both X and Y are S—S. In yet another embodiment, the organic group comprises antibodies, drugs, prodrugs, peptides, peptide fragments, amino acids, amino acid fragments, ethylene glycol units, other polymers, proteins, protein fragments, carbohydrates, carbohydrate fragments, lipids, nucleic acids, other biomolecules, synthetic molecules. In a further embodiment, the organic group is a peptide or peptide fragment. In yet a further embodiment, the organic group is an amino acid or amino acid fragment. In another embodiment, the organic group is hydrophilic. In a further embodiment, the organic group is hydrophobic. In one embodiment, is the composition having the structure of Formula (I) wherein n is an integer from 4 to about 20. In another embodiment, n is an integer from about 20 to about 100; about 100 to about 1000; and about 1000 to about 2000. In the formula (—SR₁—Au—SR₁—Au—)ₙ gold is typically in the +1 oxidation state giving the oligomer or polymer a positive charge. In other embodiments, this is accentuated, balanced, or reversed by the R₁ group used. Therefore in further embodiments, oligomers or polymers are produced that have any desired charge, or no charge at all. Because of the flexibility in design of the R₁ group, in some embodiments, the oligomers or polymers are hydrophilic or hydrophobic.

In some embodiments, the fundamental structure of these metal oligomers and polymers is also varied such that the number of interspersed organic groups is varied, e.g., (—SR₁—Au—SR₂—Au—)ₙ is also constructed with two or more intervening gold bonding atoms, such as (—SR₁S—Au—SR₂—SAu—)ₙ or (—S—S—Au—S—S—Au—)ₙ (see structures below.) In further embodiments, atoms bound to gold with three or more coordination bonds, such as phosphorus and nitrogen, are used to create branched polymers. The organic groups disclosed herein also contain linkable groups, either for covalent linking to further polymerization, or include structures that associate by non-covalent interactions, again allowing larger polymers to be formed.

One method for the formation of the metal oligomers and polymers is to mix, in appropriate solvents, and in appropriate molar ratios, a metal salt with a compound that is able to form a bond or association with the metal. In some reactions, the metal atom is oxidized or reduced to a different oxidation state. The compound complexing with the metal is an organic compound and may also be altered during formation of the oligomer or polymer. For example, in some embodiments, thiol containing compounds form metal oligomers and polymers with HAuCl₄. During the reaction, the gold atom is reduced from the +3 state to the +1 oxidation state, and the thiol loses a proton. In this case, addition of base will drive the polymerization reaction. Phosphines, in other embodiments also act as reducing agents, and reduce metals in higher oxidation states. The resultant phosphine oxide is less suitable for metal bonding, but, in further embodiments, if an excess of phosphines is used, these bond with the metal atoms. In some embodiments, addition of a driving reagent, such as a base, is required to produce the oligomer or polymer.

Oligomer and polymer size is controlled by various means. In one embodiment is a method to control the reaction concentrations and relative amounts of the starting reagents, such that individual polymers form depending on concentration, then grow until reactants are used up. In another embodiment, is a method which caps the ends of the growing polymer with a material that only has one bond available for linking, thus quenching further growth. Examples are some phosphorus compounds. Alternatively, in other embodiments, the polymerizing group is reacted with to make it unavailable for further linking to the metal. Examples are N-ethylmaleimide, other maleimide containing compounds, aziridines, acylating agents such as fluorobenzene, vinyl sulfones, iodoacetamides, and isothiocyanates that react with thiols thus blocking further linking to metal atoms. Metal-metal interactions in further embodiments are inhibited by supplying adsorbent atoms or compounds that “cap” these association sites. Examples are chelators such as ethylenediamine tetraacetic acid (EDTA), and compounds such as imidazole. The solubility of the metal oligomer or polymer, in one embodiment is different from the starting reactants, and choice of solvent is used to remove products, thus halting their further polymerization. Controlling the amount of addition of the driving reagent, such as base, in other embodiments also halts the reaction products.

Also described herein are compositions having the structure of Formula (I) wherein X and Y are P(R₃)₃. In another embodiment, X and Y are both tris-carboxyethyl phosphine, 2,2′,2″-phosphinetriyltriethanol, 3,3′,3″-phosphinetriyltripropan-1-ol, (5E,5′E,5″E,7E,7′E,7″E)-8,8′,8″-phosphinetriyltris(1,2-dihydroxyocta-5,7-dien-4-one), or (10E,12E,15E,17E)-1,27-dihydroxy-14-((1E,3E)-7-(2-(2-hydroxyethoxy)ethoxy)-5-oxohepta-1,3-dienyl)-3,6,22,25-tetraoxa-14-phosphaheptacosa-10,12,15,17-tetraene-9,19-dione. In other embodiments are compositions having the structure of Formula (I) wherein X and Y are a combination of tris-carboxyethyl phosphine, 2,2′,2″-phosphinetriyltriethanol, 3,3′,3″-phosphinetriyltripropan-1-ol, (5E,5′E,5″E,7E,7′E,7″E)-8,8′,8″-phosphinetriyltris(1,2-dihydroxyocta-5,7-dien-4-one), or (10E,12E,15E,17E)-1,27-dihydroxy-14-((1E,3E)-7-(2-(2-hydroxyethoxy)ethoxy)-5-oxohepta-1,3-dienyl)-3,6,22,25-tetraoxa-14-phosphaheptacosa-10,12,15,17-tetraene-9,19-dione.

In yet another aspect is a composition having the structure of Formula (IA):

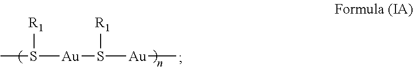

wherein each R₁ is an organic group comprising a peptide fragment, a peptide, an antibody fragment, an antibody, a single chain antibody fragment, a single chain antibody, a protein fragment, a protein, a lipid fragment, a lipid, a carbohydrate fragment, a carbohydrate, an aptamer fragment, an aptamer, a nucleic acid fragment, a nucleic acid, a thiol-containing moiety, a porphyrin fragment or a porphyrin; n is an integer from 2 to about 2000 and a pharmaceutically acceptable buffer. In another embodiment, is a composition having the structure of Formula (IA) wherein each R₁ is the same. In one embodiment, is the composition comprising the compound having the structure of Formula (IA) wherein the pharmaceutically acceptable buffer is at a concentration effective to maintain the pH of the composition within the range of about 6.5 to about 8.5. In another embodiment, the pharmaceutically acceptable buffer is at a concentration effective to maintain the pH of the composition within the range of about 7 to about 8. In a further embodiment, each R₁ is different such that the composition comprises mixed organic groups. In yet a further embodiment, the organic group is a peptide or peptide fragment. In yet another embodiment, the organic group is a protein or protein fragment. In a further embodiment, the organic group is a carbohydrate or carbohydrate fragment. In another embodiment, the organic group is a thiol-containing moiety.

In one embodiment, R₁ is an amino acid fragment, such as for example, a fragment of cysteine. In another embodiment, R₁ is cysteine such that the sulfur atom of cysteine and the sulfur atom attached to Au, forms a disulfide bond. In another embodiment, R₁ is an amino acid containing a sulfur atom. In a further embodiment, the amino acid is a naturally occurring amino acid. In yet another embodiment, the amino acid is a synthetic amino acid. In yet another embodiment, the synthetic amino acid is a non-natural amino acid. In still another embodiment, R₁ is a synthetic amino acid fragment, a non-natural amino acid fragment, or a naturally occurring amino acid fragment. In yet another embodiment, the amino acid or amino acid fragment is methionine, cysteine, or homocysteine.

Presented herein are compositions having the structure of Formula (IA) wherein R₁ is a peptide. In some embodiments, peptides or proteins containing the amino acids cysteine or histidine bind through these groups to many metal atoms and are favorable for forming metal oligomers and polymers. Similarly, in other embodiments, compounds containing thiols or histidines are used. In another embodiment, R₁ is glutathione such that the sulfur atom of glutathione and the sulfur atom attached to Au, forms a disulfide bond. In another embodiment, R₁ is a peptide containing a sulfur atom. In yet another embodiment, the peptide is a naturally occurring peptide. In a further embodiment, the peptide is a synthetic peptide. In still another embodiment, R₁ is a synthetic peptide fragment or a naturally occurring peptide fragment. In another embodiment, R₁ is a peptide or peptide fragment which contains cysteine. In one embodiment, is the composition having the structure of Formula (IA) wherein n is an integer from 4 to about 20. In another embodiment, n is an integer from about 20 to about 100; about 100 to about 1000; and about 1000 to about 2000.

Also described herein are compositions having the structure of Formula (IA) wherein R₁ is a carbohydrate or carbohydrate fragment. In one embodiment, the carbohydrate is a monosaccharide, a disaccharide, a trisaccharide, or polysaccharides such as dextran. In another embodiment, is a composition having the structure of Formula (IA) wherein R₁ is a fragment of a monosaccharide, a disaccharide or a trisaccharide. In a further embodiment is a composition having the structure of Formula (IA) wherein R₁ is a monosaccharide fragment selected from a fragment of glucose, mannose, fructose, ribose, and xylose. In another embodiment, the carbohydrate is modified with at least one sulfur atom. In another embodiment, the sulfur modified carbohydrate is attached to the metal atom via a disulfide linkage. In a further embodiment, the sulfur-containing carbohydrate is 1-thio-β-D-glucose, 5-thioglucose or 6-thioglucose.

The R₁ groups of compositions having the structure of Formula (IA) also include thiol-containing groups or fragments of thiol-containing groups such as lipoic acid, lipoamide, high molecular weight (2 to 20 kDa) PEG, thiocholesterol, thiopropionic acid, thiophenol, mercaptoethylamine, mercaptoethanol, dodecanethiol, dodecanethiol in combination with tween 20, and dithiobis[succinimidyl propionate]. In a further embodiment, the R₁ group of a composition having the structure of Formula (IA) is dithiothreitol.

The embodiments described herein also include compositions having the structure of Formula (IA) wherein the R₁ groups are different. By way of example only, in one embodiment, the composition described herein has the structure:

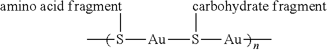

where n is an integer from 4 to about 2000. In another embodiment, by way of example only, the composition described herein has the structure:

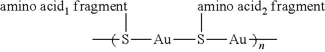

where n is an integer from 4 to about 2000, such that amino acid₁ and amino acid₂ are not the same. In another embodiment, by way of example only, the composition described herein has the structure:

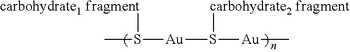

where n is an integer from about 4 to about 2000, such that carbohydrate, and carbohydrate₂ are different. Various permutations using the organic groups described previously are contemplated herein. For example, in one embodiment, the composition having the structure of Formula (IA) has alternating organic groups wherein the organic groups consist of two alternating and different amino acid fragments. In a further embodiment, the organic groups consist of two alternating peptide fragments. In further embodiments, the organic groups consist of two alternating thiol-containing groups or fragments of thiol-containing groups selected from lipoic acid, lipoamide, high molecular weight (2 to 20 kDa) PEG, thiocholesterol, thiopropionic acid, thiophenol, mercaptoethylamine, mercaptoethanol, dodecanethiol, dodecanethiol in combination with tween 20, and dithiobis[succinimidyl propionate].

Also described herein are compositions having the structure of Formula (IB):

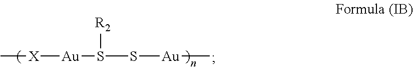

wherein X is selected from S(R₁) or S(R₂)—S, or S—S; R₁ and R₂ are each independently a peptide fragment, a peptide, an antibody fragment, an antibody, a single chain antibody fragment, a single chain antibody, a protein fragment, a protein, a lipid fragment, a lipid, a carbohydrate fragment, a carbohydrate, an aptamer fragment, an aptamer, a nucleic acid fragment, a nucleic acid, a thiol-containing moiety, a porphyrin fragment or a porphyrin; n is an integer from 2 to about 2000; and a pharmaceutically acceptable buffer.

In one embodiment, is the composition comprising the compound having the structure of Formula (IB) wherein the pharmaceutically acceptable buffer is at a concentration effective to maintain the pH of the composition within the range of about 6.5 to about 8.5. In another embodiment, the pharmaceutically acceptable buffer is at a concentration effective to maintain the pH of the composition within the range of about 7 to about 8.

In one embodiment, X is S(R₁). In another embodiment, is a composition having the structure of Formula (IB) wherein X is S(R₁) and R₁ and R₂ are the same. In a further embodiment, is a composition having the structure of Formula (IB) wherein X is S(R₁) and R₁ and R₂ are different. In one embodiment, R₁ and R₂ are each an amino acid or amino acid fragment. In another embodiment, R₁ and R₂ are each a peptide or peptide fragment. In another embodiment, R₁ and R₂ are each a protein or protein fragment. In a further embodiment, R₁ and R₂ are each a sugar or sugar fragment. In yet a further embodiment, R₁ and R₂ are each a thiol-containing moiety. In some embodiments, Au is replaced with another metal atom, such as for example, Zn or Fe. In further embodiments, one Au atom is replaced with another metal atom, such as for example, Zn, such that the composition comprises mixed metal atoms.

In another embodiment, are compositions having the structure of Formulas (IC) or (ID):

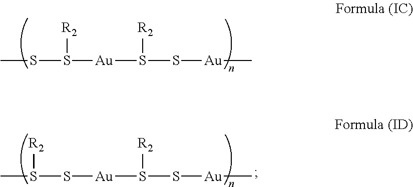

wherein each R₂ is independently a peptide fragment, a peptide, an antibody fragment, an antibody, a single chain antibody fragment, a single chain antibody, a protein fragment, a protein, a lipid fragment, a lipid, a carbohydrate fragment, a carbohydrate, an aptamer fragment, an aptamer, a nucleic acid fragment, a nucleic acid, a thiol-containing moiety, a porphyrin fragment or a porphyrin; n is an integer from 2 to about 2000; and a pharmaceutically acceptable buffer.

In one embodiment, is the composition comprising the compound having the structure of Formula (IC) or (ID) wherein the pharmaceutically acceptable buffer is at a concentration effective to maintain the pH of the composition within the range of about 6.5 to about 8.5. In another embodiment, the pharmaceutically acceptable buffer is at a concentration effective to maintain the pH of the composition within the range of about 7 to about 8.

In one embodiment, is the composition having the structure of Formulas (IC) or (ID) wherein each R₂ is the same or different. In one embodiment, R₂ is an amino acid or amino acid fragment. In another embodiment, R₂ is a peptide or peptide fragment. In another embodiment, R₂ is a protein or protein fragment. In a further embodiment, R₂ is a sugar or sugar fragment. In yet a further embodiment, R₂ is a thiol-containing moiety. R₂ groups described herein include, but are not limited to, a fragment of cysteine, a naturally occurring amino acid, a synthetic amino acid, a non-natural amino acid, a synthetic amino acid fragment, a non-natural amino acid fragment, or a naturally occurring amino acid fragment, methionine, homocysteine, glutathione, a naturally occurring peptide, a synthetic peptide, a synthetic peptide fragment, a naturally occurring peptide fragment, a peptide or peptide fragment which contains cysteine, a carbohydrate or carbohydrate fragment, a monosaccharide, a disaccharide, a trisaccharide, a fragment of a monosaccharide, a disaccharide or a trisaccharide, a fragment of glucose, mannose, fructose, ribose, or xylose, a carbohydrate modified with at least one sulfur atom, 5-thioglucose, 6-thioglucose, lipoic acid, lipoamide, high molecular weight (2 to 20 kDa) PEG, thiocholesterol, thiopropionic acid, thiophenol, mercaptoethylamine, mercaptoethanol, dodecanethiol, dodecanethiol in combination with tween 20, and dithiobis[succinimidyl propionate], dithiothreitol, or fragments of lipoic acid, lipoamide, high molecular weight (2 to 20 kDa) PEG, thiocholesterol, thiopropionic acid, thiophenol, mercaptoethylamine, mercaptoethanol, dodecanethiol, dodecanethiol in combination with tween 20, and dithiobis[succinimidyl propionate], and dithiothreitol or combinations thereof.

While compositions having the structure of Formulas (IC) or (ID) comprise R₂ groups which are the same, further compositions wherein R₂ is different are also described herein. For example, in some embodiments, the compositions having the structure of Formula (IC) or (ID) comprise R₂ groups that are amino acid fragments but differ in the specific type of amino acid fragment such as, for example, a cysteine fragment and a homocysteine fragment. Other compositions comprise different R₂ such as, by way of example only, a composition having the structure of Formula (IC) or (ID) wherein R₂ is alternating between an amino acid fragment and a peptide fragment. One embodiment is a composition having the structure of Formula (IC) or (ID) wherein R₂ alternates between a cysteine fragment and a glutathione fragment. Also disclosed herein are embodiments wherein the compositions described herein employ different organic groups such that the organic groups vary in composition pattern. For example, in one embodiment, is a composition having the structure of Formula (I), (IA), (IB), (IC), (ID), or (II) wherein the distribution of organic groups does not alternate but is random.

In further embodiments, are compositions having the structure of Formulas (IC) or (ID) wherein n is an integer from about 20 to about 100; about 100 to about 1000; and about 1000 to about 2000.

### Larger Metal Oligomers and Polymers

Also presented herein are larger metal oligomers and polymers formed from interactions between smaller oligomers/polymers. In some embodiments, metal-metal bonds are formed and/or alternatively organic-organic interactions that also lead to larger oligomers or polymers. In some embodiments, organic groups are also used to create larger structures by their interactions. For example, benzene rings are known to stack, and alkyl chains associate.

In some embodiments, the metal oligomers and/or polymers form larger metal oligomers and/or polymers via noncovalent interactions between organic compounds containing aromatic moieties, or Π-Π interactions caused by intermolecular overlapping of p-orbitals in Π-conjugated systems so they become stronger as the number of Π-electrons increases. In some embodiments are large metal oligomers and/or polymers comprised of small metal oligomers and/or polymers, wherein the organic group of each small metal oligomer and/or polymer is a nucleotide made from purine or pyrimidine rings, such that pi bonds extending from atoms of one small metal oligomer and/or polymer overlaps with pi bonds of another small metal oligomer and/or polymer, thereby forming larger metal oligomers and/or polymers.

By way of example only, small metal oligomers and polymers having the structure —(SR₁—Au—SR₁—Au)ₙ— wherein R₁ is a long chain alkyl group, associates with another small metal oligomer and polymer having a long chain alkyl group to form a larger oligomer or polymer. Particularly if the organic group is not highly soluble in a particular solvent, the organic groups will tend to aggregate, thus forming larger associated structures. Examples are alkyl chains or aryl groups that are not very soluble in polar solvents, thus forcing the organic groups to associate, thus making larger metal-organic polymers. Conversely, ionic groups are more soluble in polar media and when placed in more organic solvents will similarly self associate to form larger metal-organic polymers. An additional chemical aspect is the charge of the organic group and the metal atom.

Reducing Environment

Another facet of these metal oligomers and polymers is their behavior with reducing agents. Many of the metal oligomers and polymers described are composed of metals in various oxidation states, such as (—SR₁—Au—SR₁—Au—)ₙ, where gold is in the +1 oxidation state. Upon exposure to some reducing agents, in some embodiments, metal atoms are reduced to lower oxidation states, thus altering their bonding properties and structures. For example, if a (—SR₁—Au—SR₁—Au—)ₙ polymer is reduced with sodium borohydride, a metal nanoparticle is formed composed of a core of gold atoms in the zero oxidation state with surface gold atoms bound to —SR groups. Control of the polymer size can then control the size of the nanoparticle formed. Partial reduction in further embodiments, leads to some of the metal atoms linking together, resulting in new nanoparticle-metal oligomer or polymer constructs.

As described above, in other embodiments, the metal oligomers and polymers are further reduced or aggregated to form metal nanoparticles. This, in other embodiments, is accomplished by supplying a reducing agent or making use of the enzymes and reducing materials present in cells. For example, intracellular glutathione concentration is much higher within cells whereas it is very low in the blood. In other embodiments, metal oligomers and polymers delivered to tumors, for example by antibodies or peptides, are endocytosed and then exposed to higher reducing concentrations, thus enabling formation of metal nanoparticles. Another effect is the degradation of the metal oligomers and polymers within cells. Endosomes fuse with lysosomes and the enzymes can breakdown the organic groups incorporated into the metal oligomers and polymers. This can serve to aggregate the metal atoms. Compositions having the structure of Formulas (I), (IA), (IB), (IC), (ID) or (II) having at least one disulfide bond, (such as a disulfide bond formed from a sulfur atom of an organic group and the sulfur atom attached to the metal atom; or a disulfide bond formed from a —S—S— group bonded to the metal atom; or a disulfide bond of an organic group or linker) are able to undergo reduction to release the organic group, the metal atom, and/or small portions of the metal oligomer and/or polymer when exposed to an environment suitable for reduction. In other embodiments, the polymer or oligomer is broken down in the cell to release a drug or enable migration to other cell or body compartments, or to enhance clearance.

An additional strategy of design is to make the metal oligomers and polymers so that they contain carboxyl groups. The endosomal pH drops to 5.5 due to proton pumps in the membrane. This can cause the metal oligomers and polymers to precipitate and aggregate.

In all of these cases, the electronic and absorption properties of the metal oligomers and polymers will be altered. For example, the coloration produced by reduction or aggregation means the material is now absorptive in other wavelengths. In further embodiments, this spectral shift is used for detection. In some embodiments, infrared (IR) absorption increases mean that the metal oligomers and polymers are heated more effectively by an infrared source. Hyperthermia is also used as a therapy, in other embodiments. This would provide a highly specific heating to the target tissue, since the metal oligomers and polymers before reduction or aggregation are not absorptive, but in the targeted tissue they become very absorptive. The aggregated metals are also more sensitive to ultrasound, microwaves, and electromagnetic oscillations. Magnetic metal oligomers and polymers that become clustered will be more sensitive to alternating field heating in the radiofrequency (3 Hz to 3 GHz) and microwave (0.3 GHz to 300 GHz) range. Thus, in some instances, the increased interaction is used both for detection or imaging and for therapy.

Many applications disclosed pertain to in vivo uses for therapies. However, in further embodiments, the metal oligomers and polymers are used ex vivo to also detect, image, or ablate tissues, cells, or other materials. For example, in some embodiments, blood is treated ex vivo by irradiation applied to an extracorporeal shunt. This avoids bodily exposure to the radiation. Organs can even be removed for treatment, such as a liver or kidney, then surgically returned to the patient, also to avoid normal tissue damage. Transplants, in other embodiments, are treated before implantation to remove materials and cells that would cause rejection.

The metal oligomers and polymers are also used on biopsies or tissue sections for detection of specific antigens. The small metal oligomers and polymers described herein have good penetration and targeting properties, with detection, in some cases by reduction to particles (which are further amplified with autometallography, similar to photographic development), fluorescence, infrared absorption, and other detectable signals possible, as disclosed. The use ex vivo also expands the list of metals and organic groups that in other embodiments is used in the metal oligomers and polymers, since systemic toxicity is not an issue.

A wide variety of metal atoms, in other embodiments, is used including the alkali metals: lithium, sodium, potassium, rubidium, cesium, francium; the alkaline earth metals: beryllium, magnesium, calcium, strontium, barium, radium; the transition metals: scandium, titanium, vanadium, chromium, manganese, iron, cobalt, nickel, copper, yttrium, zirconium, niobium, technetium, ruthenium, rhodium, palladium, silver, hafnium, tantalum, tungsten, rhenium, osmium, iridium, platinum, gold, mercury, rutherfordium, dubnium, seaborgium, bohrium, hassium, meitnerium, darmstadtium, roentgenium, ununbium; the poor metals: aluminium, gallium, indium, tin, thallium, lead, bismuth, ununtrium, ununquadium, ununpentium, ununhexium, lanthanoids, lanthanum, cerium, praseodymium, neodymium, promethium, samarium, europium, gadolinium, terbium, dysprosium, holmium, erbium, thulium, ytterbium, lutetium; and the actinoids: actinium, thorium, protactinium, uranium, neptunium, plutonium, americium, curium, berkelium, californium, einsteinium, fermium, mendelevium, nobelium, and lawrencium. Some of these and their isotopes are favorable for various applications: those with unpaired electrons or that alter proton resonances can be useful for MRI (magnetic resonance imaging), such as gadolinium, manganese, dysprosium, and iron. Those with high atomic number can be useful for x-ray planar and CT (computed tomography) imaging, including but not limited to gold, platinum, osmium, iridium, thallium, lead, bismuth, tungsten, silver, palladium, and molybdenum. Metals with high cross section absorbers of various radiations are useful for capturing the radiation energy and transferring it locally to surrounding molecules or tissues. Forms of radiation include, but are not limited to, visible light, lasers, infrared, microwave, radio frequencies, ultraviolet radiation, and other electromagnetic radiation at various frequencies. Various other sources may be employed, including, but not limited to: electrons, protons, ion beams, carbon ions, and neutrons. The higher atomic number elements (Z>50) are favored for radiation absorbance and related effects. Radiation enhancement effects are useful also for improving therapies. Radioactive metals or atoms in the organic moieties of the metal oligomers and polymers can be useful for a number of purposes, such as imaging by PET (positron emission tomography) and SPECT (single photon emission computed tomography) or therapies based on delivering radioisotopes.

### Uses

Imaging

In one aspect, the oligomers and polymers demonstrate properties that are useful in applications such as medical imaging, where properties are chosen to optimize such characteristics as blood half-life, tumor, organ, or tissue targeting, and clearance. The oligomers and polymers disclosed herein also have utility as detection reporters, using for example, visible light, infrared, ultraviolet (UV), or x-rays. Another embodiment provides metal oligomers and polymers with fluorescent and phosphorescent properties, and having utility as sensitive detectors.

In one aspect is a method for biological imaging of a biological system comprising, administering to the biological system a dose of the composition comprising a compound having the structure of Formula (I):

X—Au—Y—Auₙ Formula (I)

wherein:

X and Y are each independently selected from S(R₁) or S(R₂)—S, S—S, or P(R₃)₃;

R₁ and R₂ are each independently an organic group; and

n is an integer from 2 to about 2000; and

subjecting the biological system to an imaging technique.

In one embodiment, the method for biological imaging of a biological system comprises administering to the biological system a dose of the composition comprising a compound having the structure of Formula (I) described herein and a pharmaceutically acceptable buffer.

In other embodiments, the method for biological imaging of a biological system comprises administering to the biological system a dose of the composition comprising a compound having the structure of Formula (IA), (IB), (IC), (ID), (IE), or (II). In further embodiments, the composition further comprises a pharmaceutically acceptable buffer.

The compositions provided herein include a metal oligomer or polymer that absorbs radiation. In some embodiments, the compositions provided herein include a metal oligomer or polymer that can absorb radiation selected from among x-ray radiation, infrared radiation, microwave radiation, ultrasound radiation, radiofrequencies, visible electromagnetic radiation, and/or ultraviolet radiation. In some embodiments, the compositions provided herein include a metal oligomer or polymer that absorbs x-Ray radiation and/or radiofrequencies. In other embodiments, compositions provided herein include a metal oligomer or polymer that absorbs x-ray radiation.

In some embodiments, the compositions provided herein are used in x-ray imaging. In some embodiments, the compositions provided herein are used in x-ray imaging and include gold oligomers or polymers. In some embodiments, compositions provided herein are used in computer tomography (CT). In some embodiments, compositions provided herein are used in computer tomography (CT) and include gold oligomers or polymers. In some embodiments, the compositions provided herein are used in magnetic resonance imaging (MRI). In some other embodiments, compositions provided herein are used in magnetic resonance imaging (MRI) and include gold oligomers or polymers. In other embodiments, compositions provided herein are used in medical applications as a contrast agent. In other embodiments, compositions provided herein are used in cancer therapy to increase and/or direct radiation to tumor cells. In other embodiments, compositions provided herein are used to increase the amount of radiation delivered to tissues and/or cells. In other embodiments, compositions provided herein are used to direct radiation to tissues and/or cells. In some other embodiments, compositions provided herein include a radioisotope of an element that emits radiation, such as, but not limited to, beta radiation.

Other properties of the metal oligomers and polymers make them useful for fluorescent and phosphorescent imaging, light and electron microscope imaging, and ultrasound imaging and ablation.

Another feature is the multifunctional aspect of these metal oligomers or polymers. In further embodiments, more than one metal and/or more than one organic group is included, thus endowing the product with multiple characteristics. For example, in some embodiments, atoms and organic moieties useful for MRI, planar x-ray and CT, PET, ultrasound imaging, SPECT, fluorescent and phosphorescent imaging, light and electron microscope imaging, and other imaging modalities are combined for multimodal imaging, or combining imaging properties with therapeutic aspects. In yet other embodiments, various substituent groups are mixed to include, for example, PEG, sugars, antibodies, peptides, drugs, sugar fragments, antibody fragments, peptide fragments, drug fragments, and other desired molecules, or to include reactive substituents such as carboxyl, amine, aldehyde, maleimido, hydroxy succinimide, hydrazide, and other linkable or reactive groups. In this way, multifunctional metal oligomers and polymers are formed that are targeted by several means (e.g., with two or more peptides), and in other embodiments, are combined with imaging or therapeutic modalities. In yet other embodiments, a variety of organic groups are incorporated into one metal oligomer and polymer to enhance its desired properties, such as including PEG for evasion of uptake by the reticuloendothelial system, especially the liver, a targeting peptide to deliver the oligomer or polymer to the desired site, and a drug for therapy, where the metal atoms enable visualization.

Another property of these metal oligomers and polymers is their potential for fluorescence and phosphorescence. Some of the polymers, even without the usually required resonant aryl or alkyne groups can exhibit fluorescence with little bleaching (i.e., was very stable). Because the fluorescence and phosphorescence is based on a metal rather than an organic group, in some embodiments, they are very stable with respect to damage. Because, in other embodiments, the metal oligomers and polymers are formed with aqueous organic groups, they are compatible with living material and do not need the extensive coatings required for, e.g., quantum dots. In additional embodiments, these metal oligomers and polymers are made biocompatible and of extremely low toxicity, and offer significant advantages over quantum dots which typically contain toxic cadmium or lead.

Applied Radiation

The metal oligomers and polymers described herein find use in applications of applied radiation. Many metals have favorable cross sections for capturing various forms of radiation. In one embodiment, the metal atoms themselves are utilized for this property, for example with x-rays, or the metals in combination with the oligomer or polymer are used where the interatom bonds create favorable absorbances, for example, in some embodiments, the absorption of ultraviolet, visible, and infrared is enhanced by metal-metal bonding or organic groups contained in the metal oligomers and polymers. In other embodiments, the favorable absorptions are used with many forms of incident radiation for improved detection and imaging, as well as therapies based on this increased absorption.

In the case of x-rays, high atomic number metal atoms have higher cross sections for absorption than tissue atoms, and in further embodiments, this is used to increase the radiation dose in the region as well as stimulate or produce other effects. X-rays can produce secondary electrons upon impinging on metal atoms, and this photoelectric effect predominates in the 5 to 400 keV region. The secondary electrons ejected can create additional ionizations, formation of free radicals, break chemical bonds, and thus cause damage to cells, in effect raising the dose deposition around them. In other embodiments, when the metal oligomers and polymers are targeted to a tumor or plaque or other biological site, the radiation effects will be enhanced. This effect, in some other embodiments, is used to specifically enhance radiotherapy of tumors. At higher incident x-ray energies (10-30 MeV), pair production increases and this, in some embodiments, is similarly used to enhance radiation effects. Gold is particularly favorable for use in the metal oligomers and polymers due to its low reactivity and low toxicity, although other choices include, but are not limited to: platinum, osmium, iridium, thallium, lead, bismuth, tungsten, silver, palladium, and molybdenum.

Auger electrons are also produced upon irradiation, but these low energy electrons, even though they are quite potentially damaging, only travel a short distance, for example, some about 10 nm. However, if the gold atoms are near a suitable target, in some embodiments, these Auger electrons are utilized to an advantage. In other embodiments, metal delivered to DNA can effectively damage it and sterilize the cell, thus stopping tumor growth, for example. Other targets, such as the cell membrane may also be used to inflict serious injury to cells. In further embodiments, the Auger electrons are used to create free radicals that travel longer distances and thus extend the damage range. In yet further embodiments, this effect is augmented by having a molecule that has a favorable yield of free radical production incorporated into the metal oligomer or polymer, by means previously described, namely bonded to the metal atom, bonded to the organic moiety of the oligomer or polymer, intercalated into the oligomer or polymer, or adsorbed to it. Compounds that more readily produce free radicals include “sensitizers” such as porphyrins, photophrin, texaphyrin, cyanine dyes, such as phthalocyanine, and other such molecules. In other embodiments, the metal oligomer or polymer is also bound to the DNA or other sensitive cell component, such as membranes, where the Auger electrons are able to inflict their damage directly. In yet further embodiments, DNA binding of the metal oligomers and polymers are enhanced by making them positively charged so that they bind to the negatively charged DNA, or incorporation of DNA intercalator molecules such as ethidium bromide, Hoeschst dyes, and acridines. Substances that bind to DNA such as histones, in other embodiments are also targeted. A further embodiment for use of the Auger emissions is to incorporate a therapeutic molecule that breaking a bond either activates it or releases it from the metal oligomer or polymer. Examples are 5-fluorouracil derivatives that become metabolically active (inhibiting DNA synthesis) upon irradiation that breaks one bond. Other embodiments include molecules that are activated that then perform chemical reactions. Energy collected from the irradiation by the metal is transferred to the compound, thus activating it. An example is a metal oligomer or polymer that incorporates the trifluoroaziridine group. Upon irradiation, the metal absorbs energy and transfers this via Auger electrons, secondary electrons or other means to the trifluoroaziridine group that is then activated to undergo crosslinking with nearby materials, such as cellular components. A therapeutic effect results by interfering with normal functions of the target materials or cellular components. The above effects are useful for ablating unwanted tissue such as tumors, atherosclerotic plaque, other forms of plaque such as in the central nervous system, fibotic material, scar tissue, warts, blockages, overactive nervous tissue such as causing epilepsy, heart irregularities, dementia, pain, and malformations.

Another embodiment provides mixtures with more than one metal and/or more than one organic moiety, especially useful for multifunctional purposes. Another embodiment provides metal oligomers and polymers having therapeutic utility, such as delivery of metal to a site that is then irradiated with, for example, visible light, lasers, infrared, microwave, radio frequencies, ultraviolet radiation, other electromagnetic radiation at various frequencies, and other sources, including, electrons, protons, positrons, beta particles, gamma rays, ion beams, and neutrons. Irradiated metal atoms in the oligomer and polymer in some embodiments produce scattering, absorption, secondary radiation such as electrons, Auger electrons, and photons, and these may be used to alter surrounding material, such as damage to tumor cells. Ionizations, free radicals, reactive oxygen species, and other products produced upon irradiation can be used to effect damage, crosslinking, bond breaking, drug release, drug activation, and other changes in surrounding material.

Brachytherapy is a favorable technique to combine with the metal oligomers and polymers described herein. After delivery of the metal oligomer or polymer to tumors or tissue to be ablated, radioactive “seeds” are placed in the target tissue. The radiation is then enhanced by the metal and any associated sensitzers (such as described above) that it contains. This is a very advantageous synergy, since the metal oligomers and polymers if delivered intravenously may not penetrate solid tumors uniformly, but may accumulate more at their growing edge, because this is the site of angiogenic vasculature which is more leaky. Also, many carcinomas have poor central circulation due to the increased tumor pressure. The central tumor cells are commonly radioresistant since they are hypoxic and not dividing as rapidly, and therefore less sensitive to external beam radiation. After radiotherapy, these central cells can survive and regrow the tumor. However, by placing a radioactive seed in the tumor center, the highest dose is delivered to these cells, thus well treating them. The radiation falls off as 1/r², and without the metal oligomer or polymer, this radiation may not treat the growing edge of the tumor well. By administering the metal oligomer or polymer, which, in some embodiments, is more concentrated at the growing edge, the dose is boosted so that this part of the tumor is also well-treated. A further advantage is that with normal brachytherapy, the dose pattern is roughly spherical, centered around the radioactive seed. However, in some embodiments, when tumor targeted metal oligomers or polymers are delivered, they follow the irregular tumor morphology and enable the radiation dose to then also follow the exact tumor shape. An additional benefit is that the metal absorbs radiation from the emitting seed, and thus the radiation is less outside the tumor boundary. This means there will be better sparing of normal surrounding tissue since it will receive a lower dose than without the metal oligomer or polymer. Favorable brachytherapy sources include: 125I (t1/2=60.2 days, ˜27 keV), 169Yb (t1/2=32.0 days, ˜93 keV), 103Pd (t1/2=17.2 days, 20-23 keV). The following have less metal interaction, but still lead to some dose enhancement: 192Ir (t1/2=73.7 days, ˜395 keV), 137Cs (t1/2=30.0 years), Co-60 (t1/2=5.25 years).

In one aspect is a method of brachytherapy, comprising implanting a dose of the composition comprising a compound having the structure of Formula (I):

X—Au—Y—Auₙ Formula (I)

wherein:

X and Y are each independently selected from S(R₁) or S(R₂)—S, S—S, or P(R₃)₃;

R₁ and R₂ are each independently an organic group; and

n is an integer from 2 to about 2000.

In a further embodiment, the composition further comprises an ¹²⁵I isotope, and ¹⁶⁹Yb isotope or ¹⁰³Pd isotope. In yet further embodiments, the method of brachytherapy comprises implanting a dose of a composition comprising a compound having the structure of Formula (IA), (IB), (IC), (ID), (IE), or (II).

In some embodiments, similar advantages to brachytherapy are obtained with miniature needle x-ray sources that are directly inserted into the tumor or tissue to be ablated. These small x-ray devices produce x-rays by accelerating electrons to the tip of an insertable small tube containing the target. X-rays are then generated at the tip.

In other embodiments, the enhancement and activation effects described above for x-rays are also produced by other forms of radiation, including, but not limited to: visible light, lasers, infrared, microwave, radio frequencies, ultraviolet radiation, and other electromagnetic radiation at various frequencies. Various other sources are employed in other embodiments, including, but not limited to: electrons, protons, positrons, beta particles, gamma rays, ion beams, carbon ions, and neutrons.

The metal oligomers and polymers described herein, in some embodiments, are administered intravenously, by direct injection to the site of interest, by catheterization, intraperitoneally, subcutaneously, subdermally, or orally. In further embodiments, they are delivered to target tissue either passively or actively. Angiogenic endothelium found in growing tumors is leaky compared to normal vasculature and the metal oligomers and polymers can leak out significantly and specifically into tumors via this pathway. The leak rate back into the blood is slower, as has been found for many substances and this is termed the “enhanced permeability and retention” (EPR) effect. In other embodiments, the biodistribution of metal oligomers and polymers is also controlled by incorporating influencing chemical groups such as those conferring charge, hydrophobicity, and hydrophilicity. In yet other embodiments, groups are incorporated to avoid certain tissue uptake, such as PEG which reduces uptake by the liver and spleen. The size of metal oligomer or polymer also impacts on its pharmacokinetics. Small materials may clear through the kidneys, whereas larger ones are excluded. In some embodiments, very large metal oligomers and polymers are targets for RES and macrophage uptake. In further embodiments, the metal oligomers and polymers are also targeted to a specific site by use of proteins, antibodies, antibody fragments, peptides, nucleic acids, carbohydrates, lipids, drugs, and other compounds.

In further embodiments, the metal oligomers and polymers are formed by polymerization of soluble starting reactants to insoluble products from soluble starting reactants. For example, it was found that starting with water soluble chloroauric acid and dithiothreitol, a highly insoluble material could be formed that not only was virtually insoluble in aqueous solvents, but was virtually insoluble in alcohols, methylene chloride, hexane, chloroform, tetrahydrofuran, acetone, dimethysulfoxide and dimethylformamide. This resistant polymer, and ones like it may find applications medically, for example, to implant at a tumor site so that the radiologist can use the metal absorption as a fiducial mark to perform the many fractionated irradiation treatments done on separate days accurately.

Drug Delivery

The oligomeric and/or polymeric compositions described herein find use as a platform for drug delivery. In some embodiments, therapies are enhanced by the metal oligomers and polymers disclosed herein. In some embodiments, existing drugs or existing drugs with slight chemical modification are incorporated into the metal oligomers and polymers, such as, by way of example only, by covalent linking to organic side chains of the oligomer or polymer, direct incorporation by including a linking atom, such as a sulfur group, by adsorption, by intercalation (e.g., with hydrophobic moieties), or encapsulation. In other embodiments, the drug-metal oligomer or polymer becomes a construct that has favorable new properties over the drug alone. For example, in one embodiment is a drug-metal polymer having the structure shown below:

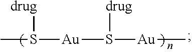

where n is an integer from 4 to about 2000. Although a 100% drug loading is shown, each sulfur need not have a drug moiety attached to it. In some embodiments, other moieties are mixed including non-functional groups or groups that impart desirable properties, such as PEG, or carbohydrates. In some embodiments, the drug is a drug fragment. In other embodiments, the composition further comprises a pharmaceutically acceptable buffer.

In other embodiments, the drug-metal polymer and/or oligomer has the structure shown below:

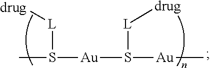

where L is a linker, and n is an integer from 4 to about 2000. In some embodiments, the drug is attached to the metal polymer via a linker that is releasable or cleavable. In some embodiments, the drug is attached to the metal polymer via a linker containing a disulfide, ester, carbamate, hydrazone or thioether moiety. In other embodiments, the linker is a readily cleavable linkage, such that it is susceptible to cleavage under conditions found in vivo. Readily cleavable linkages are, in some embodiments, linkages that are cleaved by an enzyme (e.g., an esterase, protease, phosphatase, peptidase and the like) found in or near the desired site of delivery. In further embodiments, linkers comprising disulfide bonds are severed by disulfide exchange, for example, in the presence of glutathione.

Also presented herein are drug-metal polymers and/or oligomers having the structure below:

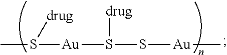

where n is an integer from 4 to about 2000. In some embodiments, the drug-metal polymer containing a disulfide bond, for example, the structure shown above, is severed by disulfide exchange, for example, in the presence of glutathione, or other reduction conditions, to release the drug.

In some embodiments, drugs suitable for use in the metal oligomer and/or polymer include, for example, chemotherapeutic agents, immunosuppressives, antibacterial agents, and antifungal agents. Anti chemotherapeutic agents, include agents such as, sulfa drugs such as salazusulfapyridine, sulfadimethoxine, sulfamethizole, sulfamethoxazole, sulfamethopyrazine and sulfamonomethoxine. Immunosuppresives, include agents such as, but are not limited to, cyclosporine such as cyclosporin A, ascomycins such as FK-506, and nonsteroidal anti-inflammatory agents such as Cox-2 inhibitors, ketorolac, suprofen, and antazoline. Other immunosuppressives include, e.g., rapamycin and tacrolimus.

Antibacterials include, e.g., beta-lactam antibiotics, such as cefoxitin, n-formamidoylthienamycin and other thienamycin derivatives, tetracyclines, chloramphenicol, neomycin, carbenicillin, colistin, penicillin G, polymyxin B, vancomycin, cefazolin, cephaloridine, chibrorifamycin, gramicidin, bacitracin, sulfonamides enoxacin, ofloxacin, cinoxacin, sparfloxacin, thiamphenicol, nalidixic acid, tosufloxacin tosilate, norfloxacin, pipemidic acid trihydrate, piromidic acid, fleroxacin, chlortetracycline, ciprofloxacin, erythromycin, gentamycin, norfloxacin, sulfacetamide, sulfixoxazole, tobramycin, and levofloxacin.

Antifungal agents include, among others, polyenes such as amphotericin B and natamycin; imidazoles such as clotrimazole, miconazole, ketoconazole, fluconazole and econazole; and pyrimidines such as flucytosine. Other exemplary antifungal agents included, e.g., itraconazole, flucytosine and pimaricin.

Antiparasitic compounds and/or anti-protozoal compounds include, e.g., ivermectin, pyrimethamine, trisulfapidimidine, clindamycin and corticosteroid preparations.

In other embodiments, drug fragments, such as for example only, fragments of immunosuppresive agents, chemotherapeutic agents, antibacterial agents, antifungal agents, and antiparasitic agents are also suitable for use as components of the metal oligomer/polymer compositions. In some embodiments, drug fragments are drugs which have been modified to enable attachment or interaction with the metal oligomer and polymer. For example, in some embodiments, the drug has been modified to incorporate a sulfur atom suitable for forming a disulfide bond with the sulfur atom of the metal oligomer and polymer. In another embodiment, the drug is modified at a position or site where activity is not required.

In further embodiments, the oligomer or polymer imparts a larger molecular weight for improved blood half-life and slower clearance, and incorporates targeting moieties, such as peptides, antibodies, antibody fragments, single chain antibodies, proteins, lipids, carbohydrates, aptamers, nucleic acids, porphyrins (many of which target tumors), and other compounds or materials. In yet other embodiments, increasing the size also enhances macrophage or other cell phagocytosis.

In some embodiments, the metal oligomers and polymers comprising a metal atom are used themselves as drugs. Varying the metal atom of the metal oligomer and polymer, in some embodiments, results in different properties. For example, silver is known to be an antimicrobial and simple gold compounds or gold nanoparticles were used to treat rheumatoid arthritis. Zinc is used in antimicrobials (e.g., bacitracin zinc, and zinc oxide is used to treat or prevent minor skin irritations, e.g., burns, cuts, poison ivy, diaper rash). It is thought that in some cases, zinc is efficacious in the treatment of (childhood) malnutrition, acne vulgaris, peptic ulcers, leg ulcers, infertility, Wilson's disease, herpes, and taste or smell disorders. Zinc has also gained popularity for its use in prevention of the common cold. Zinc is a cofactor for the antioxidant enzyme superoxide dismutase (SOD) and is in a number of enzymatic reactions involved in carbohydrate and protein metabolism. Its immunologic activities include regulation of T lymphocytes, CD4, natural killer cells, and interleukin II. Additionally, it is thought that zinc possesses antiviral activity. Further, zinc is necessary for the maturation of sperm and normal fetal development. Zinc is also involved in controlling the release of stored vitamin A from the liver. Within the endocrine system, zinc has been shown to regulate insulin activity and promote the conversion of thyroid hormones thyroxine to triiodothyronine. Thus, in some embodiments are metal oligomers and/or polymers wherein the metal is zinc, silver or gold, such that the metal is released into the body after administration.

A number of metals and metal compounds have antioxidant properties. Some tungsten compounds have antiviral activity. In some embodiments, by incorporating metals into the oligomers and polymers, their effects are modulated. For example, in other embodiments, the oligomers and polymers are biodegraded over time, allowing for controlled release of the desired metal. In further embodiments, the oligomers and polymers are also designed for more specific and targeted delivery of the metals. A number of metals are essential for life, and include Fe, Zn, Mn, Cr, Cu, Ca, and Ni. Deficiencies of metals can cause pathology, for example, iron deficiency leads to anemia, calcium deficiency to rickets and osteoporosis, zinc deficiency to growth retardation, sodium deficiency to hypo/hyper-natremia, potassium deficiency to hypo/hyper-kalemia, hypokalmia and producing irregular heartbeats, and magnesium deficiency can lead to mitral valve prolapse, migraines, attention deficit disorder, fibromyalgia, asthma and allergies. In some embodiments, the metal atom of the metal oligomers and polymers is selected from Fe, Zn, Mn, Cr, Cu, Ca, and Ni. In other embodiments, the metal atom is Zn. If further embodiments, Au.

Treatment Methods

Described herein are methods for enhancing therapies by using the metal oligomers and/or polymers described herein. In one aspect is a method for treating a subject having tumors, tumor-related disorders, and/or cancer comprising administering to a patient in need thereof a therapeutically effective amount of a composition having the structure of Formulas (I), (IA), (IB), (IC), (ID), (IE), or (II). In one embodiment, the therapeutically effective amount of a composition described herein comprises a pharmaceutically acceptable buffer. In one embodiment the tumors, tumor-related disorders, and/or cancer is selected from the group consisting of: oral cancer, prostate cancer, rectal cancer, non-small cell lung cancer, lip and oral cavity cancer, liver cancer, lung cancer, anal cancer, kidney cancer, vulvar cancer, breast cancer, oropharyngeal cancer, nasal cavity and paranasal sinus cancer, nasopharyngeal cancer, urethra cancer, small intestine cancer, bile duct cancer, bladder cancer, ovarian cancer, laryngeal cancer, hypopharyngeal cancer, gallbladder cancer, colon cancer, colorectal cancer, head and neck cancer, parathyroid cancer, penile cancer, vaginal cancer, thyroid cancer, pancreatic cancer, esophageal cancer, Hodgkin's lymphoma, leukemia-related disorders, mycosis fungoides, and myelodysplastic syndrome.

Another embodiment provides a method for treating a subject having tumors, tumor-related disorders, and/or cancer comprising administering to a patient in need thereof a therapeutically effective amount of a compound having the structure of Formula (I), (II), (IA), (IB), (IC), (ID), (IE) or (II) wherein the tumors, tumor-related disorders, and/or cancer is selected from the group consisting of: non-small cell lung cancer, pancreatic cancer, breast cancer, ovarian cancer, colorectal cancer, and head and neck cancer.

Another embodiment provides a method for treating a subject having tumors, tumor-related disorders, and/or cancer wherein the tumors, tumor-related disorders, and/or cancer is selected from the group consisting of: a carcinoma, a tumor, a neoplasm, a lymphoma, a melanoma, a glioma, a sarcoma, and a blastoma.

Immunological processes are modulated by metals and active peptides, proteins, lipids, carbohydrates, and cytokines. The metal oligomers and polymers can act as larger structure platforms for enhanced delivery or endocytosis by antigen presenting cells to improve vaccine efficiency. In some embodiments, the metal oligomers and polymers described herein are also designed to affect various cell populations, such as mast cells that are involved with allergies, B, T, and NK cells, monocytes, neutrophils, esosinophils, and basophils that are involved with various immune responses and pathologies.

Provided herein is a method for treating a subject having tumors, tumor-related disorders, and/or cancer, comprising administering to the subject, a therapeutically effective amount of a composition having the structure shown in Formula (I):

X—Au—Y—Auₙ; Formula (I)

wherein:

X and Y are each independently selected from S(R₁) or S(R₂)—S, S—S, or P(R₃)₃;

R₁ and R₂ are each independently an organic group; and n is an integer from 2 to about 2000;

wherein administration of the composition is adjunct to radiotherapy.

One embodiment provides the method of sensitizing a biological system to the effects of radiation wherein the composition of Formula (I) comprises at least one R₁, R₂, or R₃

group comprising

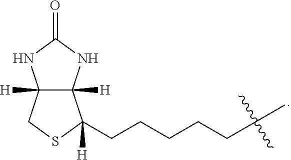

One embodiment provides the method wherein a tumor-specific antibody is non-covalently attached through a biotin-avidin complex. Another embodiment provides the method wherein the composition of Formula (I) comprises at least one R₁, R₂, or R₃ group comprising a DNA-binding moiety selected from ethidium bromide, Hoeschst dyes or acridines.

### Other Uses

In further embodiments, the metal oligomers and polymers are also included in other polymers. For example, several biodegradable polymers have been approved by the FDA and these or others, in some embodiments, incorporate metal oligomers and polymers described herein. One benefit described herein is to enhance blood half-life for improved targeting to tumors or other targets like plaque, and upon biodegradation, the metal oligomers and polymers, in other embodiments, released and clear quickly, thus reducing the whole body retention.

The metal oligomers and polymers also have many non-biological uses, for example as new substances in material science. The insoluble metal oligomers and polymers, in further embodiments, are used as impermeable coatings to protect surfaces from rust, corrosion, or other environmental insults. The heating properties are also used to polymerize or depolymerize plastics. Inclusion of metal oligomers and polymers in other materials would serve to make them visible by many other techniques such as MRI, x-ray, and fluorescence. Inclusion in money, artwork, clothing, or other store goods, in further embodiments, provide a security system with or without bar coding. The metal oligomers and polymers described herein are also used to incorporate metals into many other materials, such as plastics, cloth, liquids, and dispersants in gasses. This would enable metal sprays, conducting polymers, radiation resistant clothing or materials that shield radiation, fluorescent and phosphorescent fabrics. A silver or zinc oligomer or polymer introduced into clothing items, patches, or sprays, in other embodiments, provide antimicrobial products. Additionally, due to their small size, the metal oligomers and polymers find use in next generation electronics as nanowires, transistors, and other electrical components.

In some embodiments, the metal oligomers and polymers are colorless or lightly colored compared to nanoparticles containing the same number of metal atoms.

Properties which were unexpected were found for these metal oligomers or polymers. For example, one formed from Au⁺¹ and the tripeptide glutathione had an apparent hydrodynamic molecular weight of ˜10 KDa when run on a gel filtration column. This polymer was clear in color and was very stable. It could be dried and rerun again on the size exclusion column in physiologic buffer, phosphate buffered saline (PBS), with the same sharp peak at the same retention time. Furthermore, no toxicity was apparent at 0.4 g Au/kg when injected intravenously into mice. As mentioned above, gold nanoparticles, have disadvantages of both poor whole body clearance and discoloration of skin. For comparison, a 2 nm gold nanoparticle formed with the same ligand (glutathione) was tested. At 0.4 g Au/kg it let to skin discoloration and a whole body clearance at one week of 82%, whereas the polymer at the same gold dose led to 98% clearance. These oligomers or polymers therefore overcome the main obstacles for use of gold and other metals for human use, especially for imaging and therapy, virtually eliminating whole body retention, thus potentially long term toxicity, side effects, and interference with multiple administrations, while also removing the undesirable cosmetic effects.

### Purified Metal Oligomers/Polymers

In size exclusion chromatography, the separation of components is a function of their molecular size and the stationary phase typically does not attract the components. Separation depends on the amount of time that the substances spend in the porous stationary phase as compared to time in the fluid. In addition, the ability of a substance to permeate into pores is determined by the diffusion mobility of macromolecules which is higher for small macromolecules. Very large macromolecules may not penetrate the pores of the stationary phase at all; and, for very small macromolecules the probability of penetration is close to unity. While components of larger molecular size move more quickly past the stationary phase, components of small molecular size have a longer path length through the pores of the stationary phase and are thus retained longer in the stationary phase.

In one aspect is a purified product comprising a compound having the structure of Formula (I):

X—Au—Y—Auₙ Formula (I)

wherein:

X and Y are each independently selected from S(R₁) or S(R₂)—S, S—S, or P(R₃)₃;

R₁ and R₂ are each independently an organic group; and

n is an integer from 2 to about 2000;

wherein the compound is purified by chromatography.

In one embodiment, the compound is purified by size exclusion chromatography. In one embodiment, the size exclusion chromatography is gel filtration. In a further embodiment, the gel filtration employs a size exclusion column, such as by way of example only, Superdex 200. In another embodiment, the gel filtration is run in a phosphate buffered saline.

In one embodiment, size exclusion chromatography is used for the isolation and purification of the compounds having the structure of Formula (I), (IA), (IB), (IC), (ID), (IE) or (II). In another embodiment, a variety of stationary phases are used in size exclusion chromatography, such as dextran, cross-linked polymers of styrene-divinylbenzene, acrylamide or vinylacetate, or macroporous inorganic material, such as silica, activated charcoal, or alumina.

### EXAMPLES

The following examples are included for illustrative purposes only and are not intended to limit the scope of the invention.

### Example 1

### Formation and Characterization of Gold-Glutathione Polymers

HAuCl₄ (25 mg/ml) in water was mixed with an equimolar amount of glutathione (GSH, a tripeptide, γ-Glu-Cys-Gly) using a stock solution of 20 mg/ml in water. Water was used to reduce the gold concentration to 5 mg/ml. Other gold concentrations are also used. The pH of the solution was increased to about 8 and the solution became clear. The product was isolated on a gel filtration, size exclusion column (Superdex 200, Amersham) in phosphate buffered saline. Compared to molecular weight standards, it showed a sharp peak with an apparent molecular weight of approximately 10 kDa. This metal polymer was then rotary evaporated and found to be very soluble in aqueous buffers. Upon rechromatographing, it still showed an identical peak at approximately 10 kDa.

### Example 2

### In Vivo Compatibility of Gold-Glutathione Polymers

The gold-glutathione polymer of Example 1 was concentrated to 100 mg Au/ml in phosphate buffered saline and injected intravenously via the tail vein into mice to deliver 750 mg Au/kg body weight. The animals showed no apparent signs of toxicity and behaved normally. In addition, there was no detectable change in color in white mice after injection; it did not discolor the skin and eyes. Glutathione gold nanoparticles containing the same amount of gold injected discolored the skin and eyes.

### Example 3

### Clearance of the Gold-Glutathione Polymers

Some animals of Example 2 were sacrificed and dissected after one week, and various organs and tissues measured for gold content by atomic absorption spectroscopy. At 1 ppb sensitivity, gold was undetectable in all organs and tissues, except in the kidney, where 1-2% of the injected dose was found. Whole body retention after one week was 1-2%. Animals injected with 2 nm glutathione gold nanoparticles, using the same amount of gold, however, showed gold remaining in various tissues (liver, 5.8%, kidneys 1.4%, carcass 12.1%) and the whole body retention after one week was 19.5%. Therefore, skin discoloration and clearance were dramatically improved by the use of the metal polymer compared to a small gold nanoparticle.

### Example 4

### Formation and Characterization of Gold-Thioglucose Polymers

HAuCl₄ (25 mg/ml) in water was mixed with about three times the molar equivalent amount of thioglucose dissolved in water. Precipitation was avoided by first raising the pH of either starting material to about 8- about 12. The product was isolated on a gel filtration, size exclusion column (Superdex 200, Amersham) in phosphate buffered saline. Compared to molecular weight standards, the product showed a peak with an apparent molecular weight of about 5- about 6 kDa. This metal polymer was then rotary evaporated and found to be very soluble in aqueous buffers.

### Example 5

### In Vivo Compatibility of Gold-Thioglucose Polymers

The gold-thioglucose polymer of Example 4 was concentrated to 200 mg Au/ml in phosphate buffered saline and injected intravenously via the tail vein into mice to deliver 1 g Au/kg body weight. The animals showed no apparent signs of toxicity and behaved normally. In addition, there was no detectable change in color in white mice after injection; it did not discolor the skin and eyes, whereas 1.9 nm gold-thioglucose nanoparticles containing the same amount of gold injected turned the eyes from pink to black and significantly colored the skin black.

### Example 6

### Reduction of Gold Polymers

The gold polymers of Examples 1 and 4 were reduced with sodium borohydride and became very dark in color. Their spectra were identical to gold nanoparticles about 2 nm in size, indicating the formation of gold nanoparticles. The increased color indicated that such a reaction could be used for sensitive detection. The gold nanoparticles could be further grown in size by addition of silver or gold ions and a reducing agent, for example silver acetate and hydroquinone, thus making them many times more detectable.

### Example 7

### Formation of Gold-PEG Oligomers and Polymers

HAuCl₄ (25 mg/ml) in water was mixed with an equimolar amount of HS—CH₂—CH₂—O—CH₂—CH₂—O—CH₂—CH₂—O—CH₃ using a stock solution of 100 mg/ml in water. The pH of the solution was increased to about 8 and the solution became clear. Upon standing for several hours, an insoluble polymer formed. When isolated before precipitation by column chromatography, polymers greater than about 3 kDa were found to be stable.

HAuCl₄ (25 mg/ml) in water was mixed with an equimolar amount of HS—(CH₂—CH₂—O—)₄—H using a stock solution of 100 mg/ml in water and showed similar properties.

### Example 8

### Formation of Gold-Dithiothreitol Oligomers and Polymers

HAuCl₄ 50 mg/ml in water was mixed with an equimolar amount of dithiothreitol using a stock solution of 100 mg/ml in water. The pH of the solution was increased to about 8, and the solution became clear, and remained so for several hours, after which a white precipitate formed. If separated before precipitation by gel filtration chromatography, two peaks were discerned. The higher molecular weight material (greater than about 3 kDa) was stable, but the lower molecular weight peak (oligomers of about 400- about 2,000 Da) continued to polymerize and precipitate. This precipitate was not readily soluble in aqueous solvents, high or low pH solvents, alcohols, methylene chloride, hexane, chloroform, tetrahydrofuran, acetone, dimethysulfoxide or dimethylformamide.

### Example 9

### Formation of Other Gold-Oligomers and Polymers with Thiols

Similar to Example 1, other thiol containing compounds were found to form metal polymers. Additional compounds tested included: lipoic acid, lipoamide, high molecular weight (2 to 20 kDa) PEG, thiocholesterol, thiopropionic acid, cysteine, thiophenol, mercaptoethylamine, mercaptoethanol, dodecanethiol in combination with tween 20, and dithiobis[succinimidyl propionate].

### Example 10

### Formation of Mixed Metal Oligomers and Polymers

In order to reduce the charge of the polymer or to obtain other properties, negatively charged thiols were used in combination with uncharged thiols. HAuCl₄ (50 mg/ml) in water was mixed with 0.5 molar equivalents of lipoic acid and 0.5 molar equivalents of lipoamide, each from stock solutions of 20 mg/ml in ethanol. The pH of the solution was increased to about 8, and the solution became clear. The formation of polymers was validated by filtering through a 30 kDa molecular centrifugal filter (Millipore); reduction with sodium borohydride indicated that most of the gold was in the retentate.

### Example 11

### Formation of Metal Oligomers and Polymers with Phosphines

Similar to Example 1, except using a two fold molar excess of the phosphine, metal oligomers and polymers were formed using the phosphines, tris-carboxyethyl phosphine, P—(C₄H₄—CO—CH₂—CHOH—CH₂0H)₃, P—(CH₂CH₂OH)₃, P—(CH₂CH₂CH₂OH)₃, and P—(C₄H₄—CO—(CH₂—CH₂—O—)₂—CH₂—CH₂—OH)₃.

### Example 12

### Formation of Large Metal Polymers

HAuCl₄ (50 mg/ml) in water was mixed with 0.5 molar equivalent amount of dithiothreitol using a stock solution of 100 mg/ml in water. The pH of the solution was increased to about 8, and the solution became clear, and remained so for several hours, after which a white precipitate formed. Microscopic examination showed formation of spherical about 0.1- about 10 μm sized metal polymers.

What is claimed is:

1. A composition comprising a compound having the structure of Formula (I):
X—Au—Y—Auₙ Formula (I)
wherein:
X and Y are each independently selected from S(R₁) or S(R₂)—S, S—S, or P(R₃)₃;
R₁ and R₂ are each independently an organic group;
n is an integer from 2 to about 2000; and
a pharmaceutically acceptable buffer.

2. The composition of claim 1 having the structure of Formula (IA):

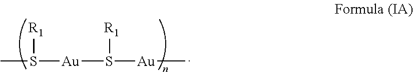

3. The composition of claim 1 having the structure of Formula (IB):

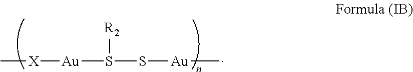

4. The composition of claim 3 wherein X is S(R₁).

5. The composition of claim 3 wherein X is S(R₂)—S.

6. The composition of claim 5 having the structure of Formulas (IC) or (ID):

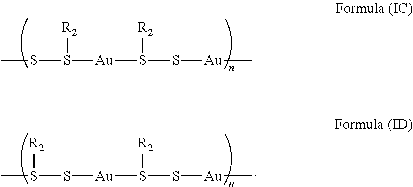

7. The composition of claim 1 wherein the organic group comprises a peptide fragment, a peptide, an antibody fragment, an antibody, a single chain antibody fragment, a single chain antibody, a protein fragment, a protein, a lipid fragment, a lipid, a carbohydrate fragment, a carbohydrate, an aptamer fragment, an aptamer, a nucleic acid fragment, a nucleic acid, a thiol-containing moiety, a porphyrin fragment or a porphyrin.

8. The composition of claim 7 wherein R₁ and/or R₂ is a peptide fragment.

9. The composition of claim 8 wherein the peptide fragment is a glutathione fragment.

10. The composition of claim 7 wherein the organic group comprises glutathione, thioglucose, dithiothreitol, lipoic acid, dihydrolipoic acid, lipoamide, dihydrolipoamide, thiocholesterol, thiopropionic acid, cysteine, thiophenol, mercaptoethylamine, mercaptoethanol, thiol-containing polyalkylene glycol, dodecanethiol in combination with tween 20, and dithiobis[succinimidyl propionate] or fragments thereof.

11. The composition of claim 1 wherein the composition has a whole body clearance of greater than about 90% after one week.

12. The composition of claim 11 wherein the composition has a whole body clearance of greater than about 95% after one week.

13. The composition of claim 1 wherein the pharmaceutically acceptable buffer is at a concentration effective to maintain the pH of the composition within the range of about 6.5 to about 8.5.

14. The composition of claim 13 wherein the pharmaceutically acceptable buffer is at a concentration effective to maintain the pH of the composition within the range of about 7 to about 8.

15. The composition of any of claim 1 wherein the pharmaceutically acceptable buffer is a phosphate buffer.

16. A method for biological imaging of a biological system comprising, administering to the biological system a dose of the composition comprising a compound having the structure of Formula (I):
X—Au—Y—Auₙ Formula (I)
wherein:
X and Y are each independently selected from S(R₁) or S(R₂)—S, S—S, or P(R₃)₃;
R₁ and R₂ are each independently an organic group; and
n is an integer from 2 to about 2000; and
subjecting the biological system to an imaging technique.

17. The method of claim 16 wherein the composition further comprises a pharmaceutically acceptable buffer.

18. The composition of claim 1 having the structure shown in Formula (IE): 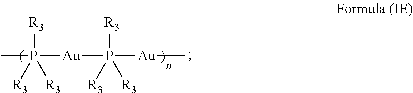
wherein each R₃ is independently an organic group.

19. The composition of claim 1 wherein the composition is preformed or formed in situ.

20. A purified product comprising a compound having the structure of Formula (I):
X—Au—Y—Auₙ Formula (I)
wherein:
X and Y are each independently selected from S(R₁) or S(R₂)—S, S—S, or P(R₃)₃;
R₁ and R₂ are each independently an organic group; and
n is an integer from 2 to about 2000; wherein the compound is purified by chromatography.

21. An injectable formulation comprising a compound having the structure of Formula (I):
X—Au—Y—Auₙ Formula (I)
wherein:
X and Y are each independently selected from S(R₁) or S(R₂)—S, S—S, or P(R₃)₃;
R₁ and R₂ are each independently an organic group; and
n is an integer from 2 to about 2000; in an amount suitable for injectable formulation.

22. The injectable formulation of claim 21 further comprising a pharmaceutically acceptable buffer.

23. The injectable formulation of claim 22 wherein the pharmaceutically acceptable buffer is a phosphate buffer.

24. The injectable formulation of claim 21 further comprising a pharmaceutically acceptable diluent or carrier.
